(19) United States
(12) Patent Application Publication
(10) Pub. No.: US 2014/0243350 A1
(43) Pub. Date: Aug. 28, 2014

(54) USE OF SEROTONIN RECEPTOR AGONISTS FOR TREATMENT OF MOVEMENT DISORDERS

(71) Applicants: John Bondo Hansen, Jyderup (DK); Mikael S. Thomsen, Hvidovre (DK)

(72) Inventors: John Bondo Hansen, Jyderup (DK); Mikael S. Thomsen, Hvidovre (DK)

(73) Assignee: CONTERA PHARMA APS, Jyderup (DK)

(21) Appl. No.: 14/126,630

(22) PCT Filed: Jul. 5, 2012
(86) PCT No.: PCT/DK2012/050254

§ 371 Date: Apr. 4, 2014

Related U.S. Application Data
(60) Provisional application No. 61/504,371, filed on Jul. 5, 2011.

(51) Int. Cl. A61K 31/506; A61K 45/06; A61K 31/44; A61K 31/27; A61K 31/216

(52) U.S. Cl. CPC A61K 31/506; A61K 31/27; A61K 31/216; A61K 31/44; A61K 45/06
USPC 514/252.15

(57) ABSTRACT

The present invention relates to the combined use of compounds which are activators of the KCNQ family potassium ion channels and compounds which are serotonin 5-HT1 receptor agonists. The combined use of KCNQ channel activators and 5-HT1 receptor agonists is useful in the treatment of for example movement disorders. The present invention further relates to pharmaceutical compositions, methods of treatments and kits of parts.

### FIELD OF INVENTION

The present invention relates to the combined use of compounds which are activators of the KCNQ family potassium ion channels and compounds which are serotonin 5-HT1 receptor agonists. The combined use of KCNQ channel activators and 5-HT1 receptor agonists is useful in the treatment of disorders and diseases such as movement disorders. The present invention further relates to pharmaceutical compositions, methods of treatments and kits of parts.

### BACKGROUND OF INVENTION

Movement disorders are a group of diseases that affect the ability to produce and control body movement, and are often associated with neurological disorders or conditions associated with neurological dysfunction. Movement disorders may manifest themselves in abnormal fluency or speed of movement, excessive or involuntary movement, or slowed or absent voluntary movement. Akathisia for example, is a movement disorder characterized by unpleasant sensations of “inner” restlessness, mental unease, or dysphoria that results in inability of a patient to sit still or remain motionless. Patients typically have restless movement, including rocking from foot to foot and walking on the spot when standing, shuffling and tramping the legs, rocking back and forth, or swinging one leg on the other when sitting. In severe cases, patients constantly pace up and down in an attempt to relieve the sense of unrest, since the restlessness is felt from wakeup in the morning to sleep at night. Some patients have described the feeling as a sense of inner tension and torment or chemical torture.

Another example of a movement disorder is dyskinesia which characterized by various involuntary movements, which can affect discrete body parts or can become generalized and severely disabling. Tardive dyskinesia is one example of dyskinesia which is characterized by repetitive, involuntary, purposeless movements, such as grimacing, tongue protrusion, lip smacking, puckering and pursing of the lips, and rapid eye blinking. Involuntary movements of the fingers may appear as though the patient is playing an invisible guitar or piano.

Often, the neurological disorder or condition which causes the movement disorder is associated with dysfunction of the basal ganglia. The dysfunction may be idiopathic, induced by certain drugs or infections, or caused by genetic defects.

Parkinson's disease (PD) is an example of a neurological disorder associated with dysfunction of the basal ganglia. PD is a common disease and affects 1% of persons above 60 years of age. PD results in movement disorders and is characterized by muscle rigidity, tremor, postural abnormalities, gait abnormalities, a slowing of physical movement (bradykinesia) and in extreme cases a loss of physical movement (akinesia). The disease is caused by progressive death and degeneration of dopamine (DA) neurons in substantia nigra pars compacta and a dysfunctional regulation of dopamine neurotransmission. In order to replace the lost dopamine, PD is currently treated with Levodopa (L-DOPA, a precursor of dopamine), with dopamine agonists or other agents that act by increasing the concentration of dopamine in the synaptic cleft. Unfortunately, the treatment of PD with L-DOPA often gives rise to dyskinesia (diminished voluntary movements and presence of involuntary movements) in advanced PD patients with impaired regulations of DA levels. This specific type of dyskinesia is called L-DOPA Induced Dyskinesia (LID) and is caused by excessive dopamine levels in the synapses (Jenner: Nat Rev Neurosci. 2008; 9(9): 665-77; Del Sorbo and Albanese: J. Neurol. 2008; 255 Suppl 4: 32-41). About 50% of patients treated with L-DOPA develop LID, which severely limits optimal treatment and reduce quality of life.

Movement disorders induced by drug therapy can also be related to treatment of other neurological or psychiatric diseases. Examples of these are tardive dyskinesia and akathesia, which are commonly developed as a side effect of long term treatment with neuroleptics for instance in patients suffering from e.g. schizophrenia.

Tardive dyskinesia may persist after withdrawal of the drug for months, years or can even be permanent. The primary prevention of tardive dyskinesia is achieved by using the lowest effective dose of a neuroleptic for the shortest time. If tardive dyskinesia is diagnosed, the therapy with the causative drug is discontinued. Both of these approaches cause difficulties for the therapeutical use of neuroleptics.

Shortly after the introduction of antipsychotic drugs in the 1950's, akathisia was recognized as one of the most common and distressing early onset adverse effects. Estimates of the prevalence of akathisia in neuroleptic-treated people range between 20% and 75%, occurring more frequently in the first three months of treatment. Akathisia is not only related to acute administration of a neuroleptic, but also to a rapid dosage increase. Unfortunately, akathisia may be difficult to distinguish from psychotic agitation or anxiety, especially if the person describes a subjective experience of akathisia in terms of being controlled by an outside force. Therefore, the dosage of the drug which causes the movement disorder may even be further increased after symptoms of akathisia.

Movement disorders are frequently caused by impaired regulation of dopamine neurotransmission. Dopamine acts by binding to synaptic dopamine receptors D1, D2, D4, and D5, and the binding is controlled by regulated release and re-uptake of dopamine. Impaired regulation of dopamine release or up-take can result in excess dopamine in the neural synapses, which lead to the development of movement disorders.

As mentioned above, PD is an example of a movement disorder associated with dysfunctional regulation of dopamine neurotransmission, which is caused by progressive degeneration of dopamine neurons. Tardive dyskinesia is another example of a movement disorder associated with dysfunctional regulation of dopamine neurotransmission. Neuroleptics act primarily on the dopamine system and are drugs which block D2 dopamine receptors; they are therefore used to prevent conditions associated with increased dopamine levels. Tardive dyskinesia has been suggested to result primarily from neuroleptic-induced dopamine super sensitivity in the nigrostriatal pathway, with the D2 dopamine receptor being most affected. Older neuroleptics, which have greater affinity for the D2 binding site, are associated with higher risks for tardive dyskinesia.

Dopamine release and re-uptake is regulated by a number of neurotransmitters, including serotonin (5-HT). Other neurotransmitters that directly or indirectly regulate dopamine neurotransmission are the inhibitory neurotransmitter gamma aminobutyric acid (GABA) and excitatory amino acid glutamate.

Serotonin acts by binding to different serotonergic receptors including the 5-HT1A, 5-HT1B, 5-HT1D, 5-HT1E, 5-HT1F, 5-HT2A, 5-HT2B, 5-HT2C, 5-HT3,5-HT4,5-HT5, 5-HT6, and 5-HT7 receptors for which both agonists and antagonists have been found. The serotonin receptors 5-HT1A, 5-HT1B, 5-HT1D, 5-HT1E, 5-HT1F are located both post-synaptically and pre-synaptically and on the cell body. Serotonin neurotransmission is regulated by these receptors and by re-uptake mechanisms (Filip et al. Pharmacol. Reports, 2009, 61, 761-777; Ohno, Central Nervous System Agents in Medicinal Chemistry, 2010, 10, 148-157).

Agonists and antagonists of some serotonergic receptors have been investigated for treatment of some movement disorders. Several serotonin 5-HT1A receptor agonists have been shown to ameliorate extrapyramidal side effects (EPS) associated with treatment with neuroleptics and to improve cognition in patients suffering from schizophrenia. (Newman-Tancredi: Current Opinion in Investigational Drugs, 2010, 11(7):802-812). In an open study relative high doses of the partial 5-HT1A receptor agonist buspirone has effects on tardive dyskinesia, Parkinsonism and akathisia in patients treated with neuroleptics. (Moss et al: J Clin Psychopharmacol. 1993, 13, 204-9).

Modulators of serotonin (5-HT) neurotransmission have been suggested to play a role in prevention of LID. However, 5-HT1A receptor agonists given in high doses can lead to the development of serotonin syndrome or serotonin toxicity a form of poisoning. The syndrome or toxicity is caused by increased activation of the 5-HT1A and 5-HT2A receptors. Serotonin syndrome, by definition, is a group of symptoms presenting as mental changes, autonomic nervous system malfunction, and neuromuscular complaints. Patients may present with confusion, agitation, diarrhoea, sweating, shivering, hypertension, fever, increased white blood cell count, incoordination, marked increase in reflexes, muscle jerks, tremor, extreme stiffness, seizures and even coma. The severity of changes ranges from mild to fatal. Because of the severity of serotonin syndrome, it is important to maintain a low exposure of the 5-HT1A receptor agonist.

### SUMMARY OF INVENTION

Surprisingly, the present inventors have found that the combined use of a KCNQ channel activator and a 5-HT1 receptor agonist effectively influences the dopamine levels in the synapse. The finding is useful in the treatment of diseases associated with altered or impaired synaptic dopamine levels such as for example movement disorders.

The combined activation of serotonergic 5-HT1 receptors and KCNQ channels can lead to a synergic effect that enables for efficacious treatment of the movement disorders as described herein. Additionally, since the combination of a KCNQ channel activator and a serotonin 5-HT1 receptor agonist provided by the present invention may allow for a reduction in dosage of the 5-HT1 receptor agonist, the KCNQ channel activator, or both, compared to known treatments, the present invention can prevent or reduce the risk of the development of serotonin syndrome and adverse effects of treatment with 5-HT1 receptor agonists as well as adverse effects of treatments with high doses of KCNQ channel activators.

The present invention relates to a pharmaceutical composition or kit of parts comprising a KCNQ channel activator and a serotonin 5-HT1 receptor agonist, or pharmaceutical acceptable derivatives thereof.

In a preferred aspect of the present invention, the pharmaceutical composition or kit of parts comprising a KCNQ channel activator and a serotonin 5-HT1 receptor agonist or pharmaceutical acceptable derivatives thereofis for use in the treatment, prevention or alleviation of movement disorders, preferably movement disorders selected from the group of akathisia, tardive dyskinesia and dyskinesia associated with Parkinson's disease, such as L-DOPA induced dyskinesia.

In another aspect the pharmaceutical composition or kit of parts comprising a KCNQ channel activator and a serotonin 5-HT1 receptor agonist or pharmaceutical acceptable derivatives thereof is for the manufacture of a medicament for the treatment of movement disorders.

A KCNQ channel activator can according to the present invention be an activator of one or more homomeric and/or heteromeric KCNQ channels each comprising one or more subunits selected from the group of Kv 7.1, Kv7.2, Kv7.3, Kv7.4 and Kv7.5, wherein KCNQ channels expressed in the neural system are preferred. A KCNQ channel activator of the present invention may be selected from the group of retigabine, flupirtine, ICA-27243, the racemic mixture BMS-204352 (maxipost), or the S enantiomer of BMS-204352, Acrylamide (S)-1, Acrylamide (S)-2, diclofenac, meclofenamic acid, NH6, zinc pyrithione and ICA-105665, wherein retigabine, flupirtine and maxipost are among the more preferred.

In one embodiment of the present invention, the 5-HT1 receptor agonist of the present invention is a compound which may or may not be a selective agonist and/or an agonist of one or more of the serotonin receptors 5-HT1A, 5-HT1B, 5-HT1D and 5-HT1F. Such 5-HT1 receptor agonist are preferably selected from the group of 5-HT1A agonists known in the art, and more preferably from the group of compounds belonging to the azapirone and/or piperazine chemical classes, such as buspirone, tandospirone and gepirone. According to the present invention, such as 5-HT1 receptor agonist can also be selected from the group of compounds being agonists of one or more of the 5-HT1B, 5-HT1D and 5-HT1F receptors, such as for example the group of triptans.

In a particular embodiment, the 5-HT1 receptor agonist is a 5-HT1A receptor agonist, such as buspirone. In a particular embodiment, the KCNQ channel activator is retigabine.

The pharmaceutical composition or kit of parts comprising a KCNQ channel activator and a serotonin 5-HT1 receptor agonist may be released or administered simultaneously, separately or sequentially.

In one embodiment of the present invention, the pharmaceutical composition or kit of parts according to the present invention comprises one or more further active ingredients, preferably selected from the group of agents which ameliorate symptoms of Parkinson's disease or which are used for treatment of Parkinson's disease, such as L-DOPA and/or DOPA decarboxylase inhibitors.

Another aspect of the present invention provides a method for treatment, prevention or alleviation of movement disorders comprising either:

a) one or more steps of administration of an effective amount of a pharmaceutical composition as defined by the present invention,
b) one or more steps of administration of an effective amount of a KCNQ channel activator and one or more steps of administration of an effective amount of a 5-HT1 receptor agonist, both compounds as defined by the present invention, or
c) one or more steps of administration as defined in both a) and b) as defined above, to an individual in need thereof.

### DEFINITIONS

An “autoreceptor” as referred to herein, is a receptor located on a pre-synaptic nerve cell and serves as a part of a feedback loop in signal transduction. It is sensitive to those neurotransmitters or hormones that are released by the neuron in whose membrane the autoreceptor sits, and functions to downregulate the release of neurotransmitters in the synapse.

The term “blood-brain barrier” refers to selective tight junctions between endothelial cells in CNS capillaries that restrict the passage of solutes into the cerebrospinal fluid (CSF).

The term “agonist” in the present context refers to a substance capable of binding to and activating a receptor. A 5-HT1A receptor agonist (5-HT1A agonist) is thus capable of binding to and activating the 5-HT1A receptor. A 5-HT1B receptor agonist (5-HT1B agonist) is capable of binding to and activating the 5-HT1B receptor. A 5-HT1D receptor agonist (5-HT1D agonist) is capable of binding to and activating the 5-HT1D receptor. A 5-HT1F receptor agonist (5-HT1F agonist) is capable of binding to and activating the 5-HT1F receptor. Said agonist compound may be an agonist of several different types of receptors, and thus capable of binding and activating several different types of receptors. Said agonist compound can also be a selective agonist which only binds and activates one type of receptor. The terms 5-HT1 agonist and 5-HT1 receptor agonist may be used interchangeably herein.

The term “antagonist” in the present context refers to a substance capable of inhibiting the effect of a receptor agonist.

The terms “dopamine,” “DA” and “4-(2-aminoethyl)benzene-1,2-diol,” refer to a catecholamine neurotransmitter and hormone. Dopamine is a precursor of adrenaline (epinephrine) and noradrenaline (norepinephrine) and activates the five types of dopamine receptors—D1, D2, D3, D4, and D5—and their variants.

A “heteroreceptor” as referred to herein, is a receptor regulating the synthesis and/or the release of mediators other than its own ligand. Heteroreceptors are presynaptic receptors that respond to neurotransmitters, neuromodulators, or neurohormones released from adjacent neurons or cells.

An “individual” in need as referred to herein, is an individual that may benefit from the administration of a combination of compounds or a pharmaceutical composition according to the present invention. Such an individual may suffer from a movement disorder or be in risk of suffering from a movement disorder. The individual may be any human being, male or female, infant or old. The movement disorder to be treated or prevented in the individual may relate to the age of the individual, the general health of the individual, the medications used for treating the individual and whether or not the individual has a prior history of suffering from diseases or disorders that may have or have induced movement disorders in the individual.

A “KCNQ channel activator” as referred to herein, is a compound capable of activating one or more voltage gated KCNQ potassium channels comprising subunits of the Kv7 family. Such activation will lead to the opening of the KCNQ channel and increase transportation of ions through the channel.

“L-DOPA” or “3,4-dihydroxyphenylalanine” is a precursor to the neurotransmitters dopamine, norepinephrine (noradrenaline), and epinephrine (adrenaline). L-DOPA is able to cross the blood-brain barrier, and is converted to dopamine by the enzyme aromatic L-amino acid decarboxylase (AADC), also known as DOPA decarboxylase (DDC). L-DOPA is used for treatment of Parkinson's disease.

A “neurotransmitter” as referred to herein, is a substance, which transmits signals from a neuron to a target cell across a neuronal synapse.

The term “Parkinson's disease,” herein abbreviated “PD” refers to a neurological syndrome characterized by a dopamine deficiency, resulting from degenerative, vascular, or inflammatory changes in the basal ganglia of the substantia nigra. Parkinson's disease is also referred to as paralysis agitans and shaking palsy.

“Partial agonists” in the present context are compounds able to bind and activate a given receptor, but having only partial efficacy at the receptor relative to a full agonist.

Partial agonists can act as antagonists when competing with a full agonist for receptor occupancy and producing a net decrease in the receptor activation compared to the effects or activation observed with the full agonist alone.

“Selective agonists” in the present context are compounds which are selective and therefore only or predominantly bind and activates one type of receptor. Thus a selective 5-HT1A receptor agonist is selective for the 5-HT1A receptor.

The term “synapse” refers to an area of a neuron that permits said neuron to pass an electrical or chemical signal to another cell. In a synapse, a plasma membrane of the signal-passing neuron (the pre-synaptic neuron) comes into close apposition with the membrane of the target (post-synaptic) cell.

The term “pharmaceutically acceptable derivative” in present context includes pharmaceutically acceptable salts or esters, which indicate a salt or ester which is not harmful to the patient. Such salts include pharmaceutically acceptable basic or acid addition salts as well as pharmaceutically acceptable metal salts, ammonium salts and alkylated ammonium salts. A pharmaceutically acceptable derivative further includes prodrugs, or other precursors of a compound which may be biologically metabolized into the active compound, or crystal forms of a compound of the present invention.

The terms “serotonin,” “5-hydroxytryptamine” and “5-HT” refers to a phenolic amine neurotransmitter produced from tryptophan by hydroxylation and decarboxylation in serotonergic neurons of the central nervous system and enterochromaffin cells of the gastrointestinal tract. Serotonin is a precursor of melatonin.

The term “terminal” in the present context refers to a neuronal terminal.

The term “therapeutically effective amount” of a compound as used herein refers to an amount sufficient to cure, alleviate, prevent, reduce the risk of, or partially arrest the clinical manifestations of a given disease or disorder and its complications. An amount adequate to accomplish this is defined as a “therapeutically effective amount”.

The terms “treatment” and “treating” as used herein refer to the management and care of a patient for the purpose of combating a condition, disease or disorder. The term is intended to include the full spectrum of treatments for a given condition from which the patient is suffering, such as administration of the active compound for the purpose of: alleviating or relieving symptoms or complications; delaying the progression of the condition, disease or disorder; curing or eliminating the condition, disease or disorder; and/or preventing the condition, disease or disorder, wherein “preventing” or “prevention” is to be understood to refer to the management and care of a patient for the purpose of hindering the development of the condition, disease or disorder, and includes the administration of the active compounds to prevent or reduce the risk of the onset of symptoms or complications. The patient to be treated is preferably a mammal, in particular a human being. Treatment of animals, such as mice, rats, dogs, cats, cows, sheep and pigs, is, however, also within the scope of the present invention. The patients to be treated according to the present invention can be of various ages.

### DESCRIPTION OF DRAWINGS

FIG. 1. The time course of effects of the KCNQ activator retigabine, the 5-HT1A agonist buspirone and combinations thereof on abnormal involuntary movements (AIM) calculated as the sum of locomotive (Lo), axial (Ax), limb (Li), and orolingual (OI) AIM scores per testing session. Asterisk mark significance levels compared to vehicle as calculated by a one-way standard ANOVA tukey post-hoc test: *P<0.05, **P<0.01 and ***P<0.001. From the curves it can be seen that retigabine alone (5 mg/kg i.p. (intraperitoneal)) did not have an effect on AIM, while buspirone alone (0.5 mg/kg i.p.) and a combination of buspirone (0.5 mg/kg i.p.) plus retigabine at lower doses (1 mg/kg i.p.) partially reduced AIM, while a combination of buspirone (0.5 mg/kg i.p.) and retigabine at higher doses (5 mg/kg i.p.) significantly reduced total AIM.

FIG. 2. The Area Under the Curves (AUC) (20-60 min) of effects of the KCNQ activator retigabine, the 5-HT1A receptor agonist buspirone and combinations thereof on abnormal involuntary movements (AIM) calculated as the sum of locomotive (Lo), axial (Ax), limb (Li), and orolingual (OI) AIM scores per testing session. Asterisk mark significance levels compared to vehicle as calculated by a one-way standard ANOVA tukey post-hoc test: *P<0.05, **P<0.01 and ***P<0.001. From the figure it can be seen that retigabine alone (5 mg/kg i.p.) did not have an effect on AIM, while buspirone alone (0.5 mg/kg i.p.) and a combination of buspirone (0.5 mg/kg i.p.) plus retigabine at lower doses (1 mg/kg i.p.) partially reduced AIM, while a combination of buspirone (0.5 mg/kg i.p.) and retigabine at higher doses (5 mg/kg i.p.) significantly reduced total AIM.

FIG. 3. The AUC (20-60 min) of effects of the KCNQ activator retigabine, the 5-HT1A agonist buspirone and combinations thereof on abnormal involuntary movements (AIM) calculated as the sum of locomotive limb (Li) AIM scores per testing session. Asterisk mark significance levels compared to vehicle as calculated by a one-way standard ANOVA tukey post-hoc test: **P<0.01 compared with vehicle, **P<0.01 compared with buspirone 0.5 mg/kg i.p. From the figure it can be seen that retigabine alone (5 mg/kg i.p.), buspirone alone (0.5 mg/kg i.p.) and a combination of buspirone (0.5 mg/kg i.p.) plus retigabine at lower doses (1 mg/kg i.p.) did not have an effect on AIMs (Li), while a combination of buspirone (0.5 mg/kg i.p.) and retigabine at higher doses (5 mg/kg i.p.) significantly reduced AIM (Li) compared to vehicle.

FIG. 4. The AUC (20-60 min) of effects of the KCNQ activator retigabine, the 5-HT1A agonist buspirone and combinations thereof on abnormal involuntary movements (AIM) calculated as the sum of locomotive Axial (Ax) AIM scores per testing session. Asterisk mark significance levels compared to vehicle as calculated by a one-way standard ANOVA Tukey post-hoc test: *P<0.05**P<0.01 compared with vehicle. From the figure it can be seen that retigabine alone (5 mg/kg i.p.) did not have an effect on AIMs (Ax), while buspirone alone (0.5 mg/kg i.p.) and a combination of buspirone (0.5 mg/kg i.p.) plus retigabine at lower doses (1 mg/kg i.p.) partially reduced Ax and a combination of buspirone (0.5 mg/kg i.p.) and retigabine at higher doses (5 mg/kg i.p.) highly significantly reduced AIM (Ax).

FIG. 5. The AUC (20-60 min) of effects of the KCNQ activator retigabine, the 5-HT1A agonist buspirone and combinations thereof on abnormal involuntary movements (AIM) calculated as the sum of Orolingual (OL) AIM scores per testing session. Asterisk mark significance levels compared to vehicle as calculated by a one-way standard ANOVA Tukey post-hoc test: *P<0.05 compared with vehicle. From the figure it can be seen that retigabine alone (5 mg/kg i.p.) and a combination of buspirone (0.5 mg/kg i.p.) plus retigabine at lower doses (1 mg/kg i.p.) did not have a significant effect on AIMs (OL), while buspirone alone (0.5 mg/kg i.p.) and a combination of buspirone (0.5 mg/kg i.p.)) plus retigabine at higher doses (5 mg/kg i.p.) significantly reduced AIM (OL).

FIG. 6. Effects of retigabine, buspirone, and combinations thereof on total move distance of naïve rats in open field test. The mean of locomotor activity in six time points during 60 minutes of rats in each group was summarized. Asterisk mark significance levels compared to vehicle as calculated by a one-way standard ANOVA tukey post-hoc test: *P<0.05, **P<0.01 and ***P<0.001. *P<0.05, **P<0.01, ***P<0. The data indicate that retigabine (10 mg/kg, i.p.) alone and retigabine (10 mg/kg i.p.) combined with buspirone (1 mg/kg i.p. or 2 mg/kg i.p.) initially significantly inhibit the locomotor activity of rats in the open field test but this effect disappears rapidly (as there are no significant differences after 20 to 60 minutes). Furthermore, the data indicate that buspirone (1 mg/kg i.p. or 2 mg/kg, i.p.) does not increase the sedative effects of retigabine.

FIG. 7. Effects of retigabine, buspirone, and combinations thereof on total velocity of naïve rats in open field test. The mean of locomotor activity in six time points during 60 minutes of rats in each group was summarized. Asterisk mark significance levels compared to vehicle as calculated by a one-way standard ANOVA tukey post-hoc test: *P<0.05, **P<0.01 and ***P<0.001. *P<0.05, **P<0.01, ***P<0. The data indicate that retigabine (10 mg/kg, i.p.) alone and retigabine (10 mg/kg i.p.) combined with buspirone (1 mg/kg i.p. or 2 mg/kg i.p.) initially significantly inhibit the locomotor activity of rats in the open field test as measured after 10 minutes but this effect disappears rapidly (as there are no significant differences after 20 to 60 minutes). Furthermore, the data indicate that buspirone (1 mg/kg i.p. or 2 mg/kg, i.p.) does not increase the sedative effects of retigabine.

FIG. 8. (A) The Area Under the Curves (AUC) (10-190 min) of effects of retigabine, buspirone and combinations thereof on total abnormal involuntary movements (AIM) calculated as the sum of locomotive (Lo), axial (Ax), limb (Li), and orolingual (OI) AIM scores per testing session. Data are expressed as mean+/−SEM, (n=6-7). The data indicate that retigabine alone (5 mg/kg s.c) did not have an effect on AIM; that buspirone alone (0.2 mg/kg s.c.) and a combination of buspirone (0.2 mg/kg s.c.) plus retigabine at lower doses (0.5 mg/kg s.c.) had a small effect on reducing AIM, while a combination of buspirone (0.2 mg/kg s.c.) and retigabine at higher doses (5 mg/kg s.c.) reduced total AIM. This combination effect was comparable if not increased when retigabine was administered 2 hours before buspirone, (B) AUC at time point 130 minutes after administration of L-DOPA.

FIG. 9. The Area Under the Curves (AUC) of effects of retigabine, buspirone and combinations thereof on individual limb (Li) AIM, axial (Ax) AIM, and orolingual (OI) AIM scores per testing session. (A) Limb AIM at 10-170 min (all time points combined) and at time point 170 min; (B) Axial AIM at 10-170 min and at time point 150 min; and (C) Orolingual AIM at 10-170 min and at time point 130 min.

### DETAILED DESCRIPTION OF THE INVENTION

The present invention relates to combinations of KCNQ channel activators and serotonin 5-HT1 receptor agonists that are able to modulate dopamine neurotransmission. Such combinations effectively suppress excessive DA neurotransmission and are therefore useful for treatment of diseases associated with altered or impaired DA regulation, such as movement disorders and preferably LID.

The KCNQ channel activator and the 5-HT1 receptor agonist may be combined in the same pharmaceutical composition, or they may be comprised in separate pharmaceutical compositions to provide a kit of parts. The KCNQ channel activator and the 5-HT1 receptor agonist may be administered or released simultaneously, separately or sequentially.

### KCNQ Channels

Ion channels are cellular proteins that regulate the flow of ions, including potassium, calcium, chloride and sodium into and out of cells. Such channels are present in all animal and human cells and affect a variety of processes including neuronal transmission, muscle contraction, and cellular secretion.

Potassium (K⁺) channels are structurally and functionally diverse families of potassium selective channel proteins, which are ubiquitous in cells, and have central importance in regulating a number of key cell functions for example in the brain, heart, pancreas, prostate, kidney, gastro-intestinal tract, small intestine and peripheral blood leukocytes, placenta, lung, spleen, colon, thymus, testis and ovaries, epithelia and inner ear organs. Humans have over 70 genes encoding potassium channel subtypes (Jentsch Nature Reviews Neuroscience 2000, 1, 21-30) with a great diversity with regard to both structure and function. While widely distributed as a class, potassium channels are differentially distributed as individual members of this class or as families.

The KCNQ channels also designated Kv7, is a voltage-dependent potassium channel family of which the genes encoding for subunits Kv7.1-Kv7.5 have currently been characterised. Mutations in four out of five Kv7 genes have been shown to underlie diseases including cardiac arrhythmias, deafness and epilepsy. All KCNQ channels share a typical topological design, consisting of a functional channel formed by four subunits; each comprising six transmembrane segments termed S1 to S6. KCQN channels can be homomers formed by the same type of subunit, or heteromers comprising different types of subunits.

A KCNQ activator is capable of binding to a KCNQ channel and triggering one or more effects, such as stabilizing the open conformation of the channel and facilitating series of conformational changes to open the channel, increased channel open times, and decreased longest closed times. As a result of these effects, the transportation of ions through the channel is increased. A number of KCNQ activating compounds have been described in the art (for example in Wulff al. Nat Rev Drug Discov. 2009 December; 8(12):982-1001 or Xiong et al. Trends Pharmacol Sci. 2008 February; 29(2):99-107, both of which are incorporated herein in their entirety). In one embodiment of the present invention, the KCNQ activator activates one or more KCNQ channels which may be homomeric or heteromeric and comprising one or more subunits selected from the group of Kv7.1, Kv7.2, Kv7.3, Kv7.4 and Kv7.5.

Neuronal KCNQ channels are distributed throughout the central nervous system and the peripheral nerves within, for example in the hippocampus, cortex, thalamus, cerebellum, brain stem and nodes of Ranvier and dorsal root ganglion neurons. Their function is primarily maintaining a negative resting membrane potential, as well as controlling membrane repolarisation following an action potential. In a preferred embodiment of the present invention, the KCNQ channel activator is activating KCNQ channels expressed in the neural system.

The KCNQ channels are differentially expressed in different parts of the brain. Here the KCNQ channels comprising Kv7.2 to Kv7.5 subunits produce the so called ‘M-current’, a low-threshold gating, slowly activating current that has profound effects on synaptic plasticity and neuronal excitability and acts as a brake for repetitive firing. One area of the brain that plays an important role in control of muscular activity and movements is the basal ganglia. Part of the basal ganglia is substantia nigra. One part of substantia nigra, the reticulata (SNr) functions similarly to the pallidum, and another part (pars compacta or SNc) provides the source of the neurotransmitter dopamine's input to the striatum.

The basal ganglia have a limbic sector whose components are assigned distinct names: the nucleus accumbens (NA), ventral pallidum, and ventral tegmental area (VTA). VTA efferents provide dopamine to the nucleus accumbens (ventral striatum) in the same way that the substantia nigra provides dopamine to the dorsal striatum.

In one embodiment of the present invention, the KCNQ activator activates one or more KCNQ channels expressed in the dopaminergic neurons of the basal ganglia, such as the substantia nigra pars compacta and/or ventral tegmental area.

Another part of the brain that plays an important role in muscular control and regulation of movements is the raphe nuclei located in the brainstem. These nuclei comprise both serotonergic and non-serotonergic neurons that send signals to several parts of the brain including the striatum, the amygdale, hippocampus, hypothalamus and neocortical regions. In one embodiment of the present invention, the KCNQ activator activates one or more KCNQ channels expressed in the raphe nuclei, such as in the serotonergic and/or non-serotonergic neurons.

In the brain, the expression of Kv7.2, Kv7.3 and Kv7.5 subunits are most abundant. The Kv7.4 subunit has the most restricted regional expression in the brain and is only present in discrete nuclei of the brainstem. Thus a KCNQ activator according to the present invention can be capable of activating one or more of the homomeric KCNQ channels comprising one type of subunit selected from the group of Kv7.2, Kv7.3, Kv7.4 and Kv7.5,

Kv7.2 subunits are capable of forming homomeric KCNQ channels formed solely by Kv7.2 subunits, but heteromerization with Kv7.3 subunits increases the M-currents, mostly due to a more efficient surface targeting and expression of functional channels. Kv7.4 subunits can also heteromerize with Kv7.3 subunits. It has been shown that these heteromers produce larger currents than homomeric Kv7.4 channels. In another embodiment of the present invention a KCNQ activator is capable of activating one or more of the heteromeric KCNQ channels, such as a KCNQ channel comprising Kv7.2 and Kv7.3 subunits, or a KCNQ channel comprising Kv7.2 and Kv7.4 subunits, or a KCNQ channel comprising Kv7.2 and Kv7.5 subunits, or a KCNQ channel comprising Kv7.3 and Kv7.4 subunits, or a KCNQ channel comprising Kv7.3 and Kv7.5 subunits, or a KCNQ channel comprising Kv7.4 and Kv7.5 subunits.

In a more preferred embodiment, the KCNQ activator activates one or more KCNQ channels selected from homomeric KCNQ channels selected from the group of KCNQ channels comprising Kv7.2, Kv7.3, Kv7.4, Kv7.5 subunits or a heteromeric KCNQ channels the selected from the group of KCNQ channels comprising Kv7.2 and Kv7.3 subunits (Kv7.2/3 channels), or comprising Kv7.3 and Kv7.4 subunits (Kv7.3/4 channels), or comprising Kv7.3 and Kv7.5 subunits (Kv7.3/5 channels).

The KCNQ channels are widely expressed at different neural subcellular locations such as somatodendritic, axonal and terminal sites. This subcellular distribution enables them to participate in both pre- and post-synaptic modulation of basal and stimulated excitatory neurotransmission.

The KCNQ channels are capable of influencing the release and neurotransmission of a number of neurotransmitters in the brain. KCNQ channels are capable of attenuating presynaptic dopaminergic neurotransmission by inhibition of basal dopamine synthesis in the presynaptic neuron, reducing accumulation of extracellular dopamine following acute blockade of striatal dopamine reuptake, and inhibition of the depolarization-induced dopamine release into the synapse. KCNQ channels have further been associated with an influence on the release of other neurotransmitters including noradrenaline, glutamate, gamma-aminobutyric acid (GABA) and acetylcholine. Thus in one embodiment of the present invention, a KCNQ channel activator is a compound capable of mediating the above mentioned functions of the KCNQ channels.

Without being bound by theory, it is suggested that KCNQ channel activators are capable of activating somatodendritic and presynaptic KQCN channels, which leads to an attenuated presynaptic dopaminergic neurotransmission that can reduce terminal synthesis and release of dopamine. Thus in one embodiment of the present invention, a KCNQ activator is an activator of one or more pre-synaptic, somatodendritic or post-synaptic KCNQ channels and preferably an activator of one or more pre-synaptic or somatodendritic KCNQ channels.

In one embodiment of the present invention, a KCNQ channel activator such as those described in the art and commercially available is used, for example retigabine (N-(2-amino-4-(4-fluorobenzylamino)-phenyl carbamic acid) ethyl ester), flupirtine (ethyl-(2-amino-6-[(4-fluorobenzyl)amino]pyridin-3-yl)carbamate), ICA-27243 (N-(6-chloro-pyridin-3-yl)-3,4-difluoro-benzamide), the racemic mixture of BMS-204352 (Maxipost, ((R/S)-(5-Chloro-2-methoxyphenyl)-3-fluoro-6-(trifluoromethyl)-2,3-dihydro-1H-indol-2-one[(R)-3-(5-chloro-2-methoxyphenyl)-3-fluoro-6-(trifluoromethyl)-1,3-dihydro-2H-indole-2-one])), the S enantiomer of BMS-204352 (S)-(5-Chloro-2-methoxyphenyl)-3-fluoro-6-(trifluoromethyl)-2,3-dihydro-1H-indol-2-one[(R)-3-(5-chloro-2-methoxyphenyl)-3-fluoro-6-(trifluoromethyl)-1,3-dihydro-2H-indole-2-one]), substituted pyridines such as those described in WO 2006092143 and WO 2011026890 (both of which are incorporated by reference herein), acryl amide (S)-1, acryl amice (S)-2, N-phenylanthralinic acid derivatives such as diclofenac, flufenamic acid, meclofenamic acid, NH6, and niflumic acid, L-364373, zinc pyrithione (bis(1-hydroxy-2(1H)-pyridineselonato-O,S) zinc) or ICA-105665 or pharmaceutically acceptable derivatives thereof.

In another embodiment of the present invention, the KCNQ channel activator is activating KCNQ channels comprising subunits selected from the group of Kv7.2, Kv7.3, Kv7.4 and Kv7.5. Thus, the KCNQ channel activator is a compound selected from the group of retigabine (N-(2-amino-4-[fluorobenzylamino]-phenyl)carbamic acid ester), flupirtine, ICA-27243 (N-(6-chloro-pyridin-3-yl)-3,4-difluoro-benzamide), the racemic mixture BMS-204352 (Maxipost) ((3S)-(+)-(5-chloro-2-methoxyphenyl)-1,3-dihydro-3-fluoro-6-(trifluoromethyl)-2H-indol-2-one), or individual R or S enantiomers of BMS-204352, Acrylamide (S)-1 ((S)—N-[1-(3-morpholin-4-yl-phenyl)-ethyl]-3-phenyl-acrylamide), Acrylamide (S)-2, diclofenac, meclofenamic acid, NH6, zinc pyrithione and ICA-105665.

In a specific embodiment of the present invention, the KCNQ channel activator is retigabine, flupirtine and/or maxipost or a pharmaceutically acceptable derivative thereof.

High doses of KCQN channel activators have been associated with adverse effects such as dizziness, headache, asthenia, nausea, somnolence, chills, pain, symptomatic hypotension, myaligia, sweating and vomiting. In one embodiment of the present invention, the combinations of a KCQN channel activator and a 5-HT1A receptor agonist allows for the use of doses which are therapeutically effective and which decrease the risk of development of adverse effects of KCQN channel activators.

### 5-HT1 Receptors

Serotonin, or 5-Hydroxytryptamine (5-HT), is a neurotransmitter that has important functions in the central nervous system of humans and animals. Serotonin has been found to regulate mood, appetite, sleep, muscle contraction, and some cognitive functions including memory and learning. Serotonin acts by binding to different serotonergic receptors, also known as 5-HT receptors. These are a group of G protein-coupled receptors (GPCRs) and ligand-gated ion channels (LGICs) found in the central and peripheral nervous systems. The 5-HT receptors include the 5-HT1A, 5-HT1B, 5-HT1D, 5-HT1E, 5-HT1F, 5-HT2A, 5-HT2B, 5-HT2C, 5-HT3,5-HT4,5-HT5,5-HT6, and 5-HT7 receptors for which both agonists and antagonists have been found.

The serotonin 5-HT1 receptors is a subfamily of 5-HT receptors including the 5-HT1A, 5-HT1B, 5-HT1D, 5-HT1E, and 5-HT1F receptors, which are G protein-coupled receptors (GPCRs) that mediate inhibitory neurotransmission. 5-HT1 receptors are located post-synaptically, pre-synaptically and on the cell body of the neurons in the cerebral cortex, hippocampus, septum, amygdale, raphe nuclei, basal ganglia and thalamus. Due to their inhibitory roles in neurotransmission, the 5-HT1 receptors play an important role in regulation of dopamine release, particularly in serotonergic (5-HT) neurons.

The 5-HT1 receptors are particularly important in the regulation of PD and associated movement disorders. In progressed PD there is extensive degenerative loss of DA neurons in substantia nigra. Transformation of L-DOPA to dopamine takes place in the remaining dopamine neurons and in 5-HT neurons, which have been shown to be able to metabolize L-DOPA to dopamine and store and release dopamine. However, serotonin neurons lack a pre-synaptic feedback control mechanism for the release of dopamine, such as the dopamine transporter and D2 auto-receptor and are therefore unable to regulate release of dopamine in a normal way. This leads to impaired levels of DA in the synapse and movement disorders

5-HT1A receptors are widely distributed in the CNS. They are principally located in the hippocampus, cingulated end enthorhinal cortices, lateral septum and mesencephalic raphe nucleus. The 5-HT1A receptors are involved in motor behavior, copulatory behavior, pain perception, emotional behavior, and cognitive processes. The 5-HT1A receptors are autoreceptors in the raphe nuclei where they are located on the cell bodies or dendrites of 5-HT neurons, or they are post-synaptic receptors. In general, activation of 5-HT1A receptors reduces the release of neurotransmitters such as 5-HT and the excitatory amino acid glutamate, which further leads to changes in dopamine release.

The 5-HT1B receptor is highly expressed in the basal ganglia and the frontal cortex. They function as autoreceptors on the terminals of 5-HT neurons inhibiting 5-HT release, or as terminal heteroreceptors on gamma-amino butyric acid (GABA), acetylcholine (Ach) and glutamate neurons where they control the release of these neurotransmitters.

The 5-HT1D receptor is present both pre-synaptically and post-synaptically in the CNS and in the periphery. The highest expression of 5-HT1D receptors in the rat brain has been found in the basal ganglia (particularly in the substantia nigra, globus pallidus and caudate putamen), the hippocampus and the cortex, while in the human brain in the basal ganglia (the substantia nigra, globus pallidus), the midbrain (the periaqueductal grey) and the spinal cord. 5-HT1D receptors are either autoreceptors on the terminals of 5-HT neurons (they inhibit 5-HT release) or terminal heteroreceptors on gamma amino butyric acid (GABA), acetylcholine (Ach) and glutamate neurons (they control the release of these neurotransmitters). 5-HT1D receptors have been described as being involved in pain perceptions and 5-HT1D receptor agonists have been developed as treatment of migraine.

The 5-HT1F receptor has been found in several CNS areas (the dorsal raphe nucleus, hippocampus, cingulate and entorhinal cortices, claustrum, caudate nucleus, brainstem) and—based on localization—suggested to function as an autoreceptor. Some triptans show high affinity for the 5-HT1F receptors.

### 5-HT1 Receptor Agonists

The present invention relates to combinations of KCNQ channel activators with 5-HT1 receptor agonists.

In one embodiment, of the present invention, the 5-HT1 receptor agonist of the present invention is a compound which may or may not be a selective agonist and an agonist of one or more of the serotonin receptors 5-HT1A, 5-HT1B, 5-HT1D and/or 5-HT1-D.

Such an agonists may be compounds binding and activating the 5-HT1A receptor, or such agonists may be compounds binding and activating the 5-HT1B receptor, or such agonists may be compounds binding and activating the 5-HT1D receptor, or such agonists may be compounds binding and activating the 5-HT1F receptor, or such an agonists may be compounds binding and activating the 5-HT1A receptor and the 5-HT1B receptor, or compounds binding and activating the 5-HT1A receptor and the 5-HT1D receptor, or compounds binding and activating the 5-HT1A receptor and the 5-HT1F receptor, or compounds binding and activating the 5-HT1A receptor and the 5-HT1B receptor and the 5-HT1D receptor, or compounds binding and activating the 5-HT1A receptor and the 5-HT1B receptor and the 5-HT1F receptor, or compounds binding and activating the 5-HT1A receptor and the 5-HT1D receptor and the 5-HT1F receptor, or compounds binding and activating the 5-HT1A, 5-HT1B, 5-HT1D and the 5-HT1F receptors.

Compounds according to the present invention which are capable of binding and activating several 5-HT1 receptors can have different affinities and/or different receptor activation efficacy for different 5-HT1 receptors, wherein affinity refers to the number and size of intermolecular forces between a ligand and its receptor, and residence time of a ligand at its receptor binding site, and receptor activation efficacy refers to the ability of the compound to produce a biological response upon binding to the target receptor and the quantitative magnitude of this response. Such differences in affinity and receptor activation efficacy can be determined by receptor binding/activation studies which are conventional in the art, for instance by generating EC₅₀ and Emax values for stimulation of [³⁵S]-GTPγS binding in cells expressing one or several types of 5-HT1 receptors as mentioned herein, or on tissues expressing the different types of 5-HT receptors. High affinity means that a lower concentration of a compound is needed to obtain a binding of 50% of the receptors compared to compounds which have lower affinity; high receptor activation efficacy means that a lower concentration of the compound is needed to obtain a 50% receptor activation response (low EC₅₀ value), compared to compounds which have lower affinity and/or receptor activity efficacy (higher EC₅₀ value).

In one specific embodiment of the present invention the 5-HT1 receptor agonist is a serotonin 5-HT1A receptor agonist (5-HT1A agonists). Such 5-HT1A receptor agonists may be partial or may not be partial agonists of the 5-HT1A receptor. The 5-HT1A receptor agonists may be selected from the group consisting of alnespirone ((+)-4-dihydro-2H-chromen-3-yl]-propylamino]butyl]-8-azaspiro[4.5]decane-7,9-dione), binospirone (8-[2-(2,3-dihydro-1,4-benzodioxin-2-ylmethylamino)ethyl]-8-azaspiro[4.5]decane-7,9-dione), buspirone (8-[4-(4-pyrimidin-2-ylpiperazin-1-yl)butyl]-8-azaspiro[4.5]decane-7,9-dione), gepirone (4,4-dimethyl-1-[4-(4-pyrimidin-2-ylpiperazin-1-yl)butyl]piperidine-2,6-dione), ipsapirone (9,9-dioxo-8-[4-(4-pyrimidin-2-ylpiperazin-1-yl)butyl]-9λ6-thia-8-azabicyclo[4.3.0]nona-1,3,5-trien-7-one), perospirone (3aR,7aS)-2-{4-[4-(1,2-benzisothiazol-3-yl)piperazin-1-yl]butyl}hexahydro-1H-isoindole-1,3(2H)-dione, tandospirone ((1R,2R,6S,7S)-4-{4-[4-(pyrimidin-2-yl)piperazin-1-yl]butyl}-4-azatricyclo[5.2.1.02,6]decane-3,5-dione), befiradol (F-13,640) (3-chloro-4-fluorophenyl-[4-fluoro-4-([(5-methylpyridin-2-yl)methylamino]methyl)piperidin-1-yl]methanone, repinotan ((R)-(−)-2-[4-[(chroman-2-ylmethyl)-amino]-butyl]-1,1-dioxo-benzo[d]isothiazolone), piclozotan (3-chloro-4-[4-[4-(2-pyridinyl)-1,2,3,6-tetrahydropyridin-1-yl]butyl]-1,4-benzoxazepin-5(4H)-one), osemozotan (5-(3-[((2S)-1,4-benzodioxan-2-ylmethyl)amino]propoxy)-1,3-benzodioxole), flesinoxan (4-fluoro-N-[2-[4-[(3S)-3-(hydroxymethyl)-2,3-dihydro-1,4-benzodioxin-8-yl]piperazin-1-yl]ethyl]benzamide), flibanserin (1-(2-{4-[3-(trifluoromethyl)phenyl]piperazin-1-yl}ethyl)-1,3-dihydro-2H-benzimidazol-2-one), sarizotan (EMD-128,130) (1-[(2R)-3,4-dihydro-2H-chromen-2-yl]-N-([5-(4-fluorophenyl)pyridin-3-yl]methyl)methanamine) or a pharmaceutically acceptable derivative thereof.

In one embodiment of the present invention, a 5-HT1A agonist is a compound of the azapirone and/or piperazine chemical classes. Such classes include buspirone tandospirone and gepirone. In a preferred embodiment of the present invention, the 5-HT1A receptor agonist is buspirone, tandospirone or gepirone.

Several 5-HT1A/5-HT1B receptor agonists are known in the art. Thus, in one embodiment of the present invention, the 5-HT1 receptor agonist is a 5-HT1A receptor agonist and a 5-HT1B receptor agonist as known in the art, such as a compound selected from the group of eltroprazine (DU-28,853), fluprazine and batoprazine.

Certain mixed 5-HT1B/5-HT1D receptor agonists have been developed, and a subgroup of 5-HT1B/5-HT1D receptor agonists are collectively called “the triptans”. The triptans have been developed as medication for treatment of migraine and have been used for therapy for more than a decade. These compounds include sumatriptan, zolmitriptan, rizatripan, naratripan, almotriptan, frovatriptan and eletriptan. In addition to their effects on 5-HT1B and 5-HT1D receptors, some “triptans” bind to and activate 5-HT1F receptors and other 5-HT receptors.

The combined 5-HT1 receptor agonist of two or more of the 5-HT1B, 5-HT1D and 5-HT1F receptors according to the present invention may be selected from the group of sumatriptan (1-[3-(2-dimethylaminoethyl)-1H-indol-5-yl]-N-methyl-methanesulfonamide), zolmitriptan ((S)-4-({3-[2-(dimethylamino)ethyl]-1H-indol-5-yl}methyl)-1,3-oxazolidin-2-one), rizatripan (N,N-dimethyl-2-[5-(1H-1,2,4-triazol-1-ylmethyl)-1H-indol-3-yl]ethanamine), naratripan (N-methyl-2-[3-(1-methylpiperidin-4-yl)-1H-indol-5-yl]ethanesulfonamide), almotriptan (N,N-dimethyl-2-[5-(pyrrolidin-1-ylsulfonylmethyl)-1H-indol-3-yl]-ethanamine), frovatriptan ((+)-(R)-3-methylamino-6-carboxamido-1,2,3,4-tetrahydrocarbazole) and eletriptan ((R)-3-[(-1-methylpyrrolidin-2-yl)methyl]-5-(2-phenylsulfonylethyl)-1H-indole) or a pharmaceutically acceptable derivative thereof.

In one particular embodiment of the present invention, the 5-HT1 receptor agonist is a 5-HT1A receptor agonist or a pharmaceutically acceptable derivative thereof and is combined with a KCNQ channel activator. Thus, in one embodiment of the present invention retigabine is used in combination with alnespirone, or retigabine is used in combination with binospirone, or retigabine is used in combination with buspirone, or retigabine is used in combination with gepirone, or retigabine is used in combination with ipsapirone, or retigabine is used in combination with perospirone, or retigabine is used in combination with tandospirone, or retigabine is used in combination with befiradol, or retigabine is used in combination with repinotan, or retigabine is used in combination with piclozotan, or retigabine is used in combination with osemozotan, or retigabine is used in combination with flesinoxan, or retigabine is used in combination with flibanserin, or retigabine is used in combination with sarizotan, or flupirtine is used in combination with alnespirone, or flupirtine is used in combination with binospirone, or flupirtine is used in combination with buspirone, or flupirtine is used in combination with gepirone, or flupirtine is used in combination with ipsapirone, or flupirtine is used in combination with perospirone, or flupirtine is used in combination with tandospirone, or flupirtine is used in combination with befiradol, or flupirtine is used in combination with repinotan, or flupirtine is used in combination with piclozotan, or flupirtine is used in combination with osemozotan, or flupirtine is used in combination with flesinoxan, or flupirtine is used in combination with flibanserin, or flupirtine is used in combination with sarizotan, or ICA-27243 is used in combination with alnespirone, or ICA-27243 is used in combination with binospirone, or is used in combination with buspirone, or ICA-27243 is used in combination with gepirone, or ICA-27243 is used in combination with ipsapirone, or ICA-27243 is used in combination with perospirone, or ICA-27243 is used in combination with tandospirone, or ICA-27243 is used in combination with befiradol, or ICA-27243 is used in combination with repinotan, or ICA-27243 is used in combination with piclozotan, or ICA-27243 is used in com-bination with osemozotan, or ICA-27243 is used in combination with flesinoxan, or ICA-27243 is used in combination with flibanserin, or ICA-27243 is used in com-bination with sarizotan, or maxipost is used in combination with alnespirone, or maxipost is used in combination with binospirone, or maxipost is used in combination with buspirone, or maxipost is used in combination with gepirone, or maxipost is used in combination with ipsapirone, or maxipost is used in combination with perospirone, or maxipost is used in combination with tandospirone, or maxipost is used in com-bination with befiradol, or maxipost is used in combination with repinotan, or Maxipost is used in combination with piclozotan, or maxipost is used in combination with osemozotan, or maxipost is used in combination with flesinoxan, or maxipost is used in combination with flibanserin, or maxipost is used in combination with sarizotan or the S enantiomer of BMS-204352 is used in combination with alnespirone, or the S enantiomer of BMS-204352 is used in combination with binospirone, or the S enan-tiomer of BMS-204352 is used in combination with buspirone, or the S enantiomer of BMS-204352 is used in combination with gepirone, or the S enantiomer of BMS-204352 is used in combination with ipsapirone, or the S enantiomer of BMS-204352 is used in combination with perospirone, or the S enantiomer of BMS-204352 is used in combination with tandospirone, or the S enantiomer of BMS-204352 is used in combination with befiradol, or the S enantiomer of BMS-204352 is used in combination with repinotan, or the S enantiomer of BMS-204352 is used in combination with piclozotan, or the S enantiomer of BMS-204352 is used in combination with osemozotan, or the S enantiomer of BMS-204352 is used in combination with flesinoxan, or the S enantiomer of BMS-204352 is used in combination with flibanserin, or the S enantiomer of BMS-204352 is used in combination with sarizotan, or Acrylamide (S)-1 is used in combination with alnespirone, or Acrylamide (S)-1 is used in combination with binospirone, or Acrylamide (S)-1 is used in combination with buspirone, or Acrylamide (S)-1 is used in combination with gepirone, or Acrylamide (S)-1 is used in combination with ipsapirone, or Acrylamide (S)-1 is used in combination with perospirone, or Acrylamide (S)-1 is used in combination with tandospirone, or Acrylamide (S)-1 is used in com-bination with befiradol, or Acrylamide (S)-1 is used in combination with repinotan, or Acrylamide (S)-1 is used in combination with piclozotan, or Acrylamide (S)-1 is used in combination with osemozotan, or Acrylamide (S)-1 is used in combination with flesinoxan, or Acrylamide (S)-1 is used in combination with flibanserin, or Acrylamide (S)-1 is used in combination with sarizotan, or Acrylamide (S)-2 is used in combination with alnespirone, or Acrylamide (S)-2 is used in combination with binospirone, or Acrylamide (S)-2 is used in combination with buspirone, or Acrylamide (S)-2 is used in combination with gepirone, or Acrylamide (S)-2 is used in combination with ipsapirone, or Acrylamide (S)-2 is used in combination with perospirone, or Acrylamide (S)-2 is used in combination with tandospirone, or Acrylamide (S)-2 is used in com-bination with befiradol, or Acrylamide (S)-2 is used in combination with repinotan, or Acrylamide (S)-2 is used in combination with piclozotan, or Acrylamide (S)-2 is used in combination with osemozotan, or Acrylamide (S)-2 is used in combination with flesinoxan, or Acrylamide (S)-2 is used in combination with flibanserin, or Acrylamide (S)-2 is used in combination with sarizotan, or DIDS is used in combination with alnespirone, or DIDS is used in combination with binospirone, or DIDS is used in combination with buspirone, or DIDS is used in combination with gepirone, or DIDS is used in combination with ipsapirone, or DIDS is used in combination with perospirone, or DIDS is used in combination with tandospirone, or DIDS is used in com-bination with befiradol, or DIDS is used in combination with repinotan, or DIDS is used in combination with piclozotan, or DIDS is used in combination with osemozotan, or DIDS is used in combination with flesinoxan, or DIDS is used in combination with flibanserin, or DIDS is used in combination with sarizotan, or diclofenac is used in combination with alnespirone, or diclofenac is used in combination with binospirone, or diclofenac is used in combination with buspirone, or diclofenac is used in combination with gepirone, or diclofenac is used in combination with ipsapirone, or diclofenac is used in combination with perospirone, or diclofenac is used in combination with tandospirone, or diclofenac is used in com-bination with befiradol, or diclofenac is used in combination with repinotan, or diclofenac is used in combination with piclozotan, or diclofenac is used in combination with osemozotan, or diclofenac is used in combination with flesinoxan, or diclofenac is used in combination with flibanserin, or diclofenac is used in combination with sarizotan, or flufenamic acid is used in combination with alnespirone, or flufenamic acid is used in combination with binospirone, or flufenamic acid is used in combination with buspirone, or flufenamic acid is used in combination with gepirone, or flufenamic acid is used in combination with ipsapirone, or flufenamic acid is used in combination with perospirone, or flufenamic acid is used in combination with tandospirone, or flufenamic acid is used in com-bination with befiradol, or flufenamic acid is used in combination with repinotan, or flufenamic acid is used in combination with piclozotan, or flufenamic acid is used in combination with osemozotan, or flufenamic acid is used in combination with flesinoxan, or flufenamic acid is used in combination with flibanserin, or flufenamic acid is used in combination with sarizotan, or meclofenamic acid is used in combination with alnespirone, or meclofenamic acid is used in combination with binospirone, or meclofenamic acid is used in combination with buspirone, or meclofenamic acid is used in combination with gepirone, or meclofenamic acid is used in combination with ipsapirone, or meclofenamic acid is used in combination with perospirone, or meclofenamic acid is used in combination with tandospirone, or meclofenamic acid is used in com-bination with befiradol, or meclofenamic acid is used in combination with repinotan, or meclofenamic acid is used in combination with piclozotan, or meclofenamic acid is used in combination with osemozotan, or meclofenamic acid is used in combination with flesinoxan, or meclofenamic acid is used in combination with flibanserin, or meclofenamic acid is used in combination with sarizotan, or mefenamic acid is used in combination with alnespirone, or mefenamic acid is used in combination with binospirone, or mefenamic acid is used in combination with buspirone, or mefenamic acid is used in combination with gepirone, or mefenamic acid is used in combination with ipsapirone, or mefenamic acid is used in combination with perospirone, or mefenamic acid is used in combination with tandospirone, or mefenamic acid is used in com-bination with befiradol, or mefenamic acid is used in combination with repinotan, or mefenamic acid is used in combination with piclozotan, or mefenamic acid is used in combination with osemozotan, or mefenamic acid is used in combination with flesinoxan, or mefenamic acid is used in combination with flibanserin, or mefenamic acid is used in combination with sarizotan, or NH6 is used in combination with alnespirone, or NH6 is used in combination with binospirone, or NH6 is used in combination with buspirone, or NH6 is used in combination with gepirone, or NH6 is used in combination with ipsapirone, or NH6 is used in combination with perospirone, or NH6 is used in combination with tandospirone, or NH6 is used in com-bination with befiradol, or NH6 is used in combination with repinotan, or NH6 is used in combination with piclozotan, or NH6 is used in combination with osemozotan, or NH6 is used in combination with flesinoxan, or NH6 is used in combination with flibanserin, or NH6 is used in combination with sarizotan, or niflumic acid is used in combination with alnespirone, or niflumic acid is used in combination with binospirone, or niflumic acid is used in combination with buspirone, or niflumic acid is used in combination with gepirone, or niflumic acid is used in combination with ipsapirone, or niflumic acid is used in combination with perospirone, or niflumic acid is used in combination with tandospirone, or niflumic acid is used in com-bination with befiradol, or niflumic acid is used in combination with repinotan, or niflumic acid is used in combination with piclozotan, or niflumic acid is used in combination with osemozotan, or niflumic acid is used in combination with flesinoxan, or niflumic acid is used in combination with flibanserin, or niflumic acid is used in combination with sarizotan, or or L-364373 is used in combination with alnespirone, or L-364373 is used in combination with binospirone, or L-364373 is used in combination with buspirone, or L-364373 is used in combination with gepirone, or L-364373 is used in combination with ipsapirone, or L-364373 is used in combination with perospirone, or L-364373 is used in combination with tandospirone, or L-364373 is used in com-bination with befiradol, or L-364373 is used in combination with repinotan, or L-364373 is used in combination with piclozotan, or L-364373 is used in combination with osemozotan, or L-364373 is used in combination with flesinoxan, or L-364373 is used in combination with flibanserin, or L-364373 is used in combination with sarizotan.

In a preferred embodiment, the KCNQ channel activator is selected from the group of retigabine, flupirtine and maxipost and the 5-HT1A receptor agonist is selected from the group of buspirone, gepirone or tandospirone.

In an even more preferred embodiment, the KCNQ channel activator is retigabine and the 5-HT1A receptor agonist is buspirone.

### Movement Disorders

The present invention relates to treatment of movement disorders, such as disorders which are associated with altered or impaired synaptic dopamine levels.

Movement disorders according to the present invention may be selected from the group of disorders comprising ataxia, akathisia, dystonia, essential tremor, Huntington's disease, myoclonus, Parkinson's disease, Rett syndrome, tardive dyskinesia, bradykinesia, akinesia, Tourette syndrome, Wilson's disease, dyskinesia, chorea, Machado-Joseph disease, restless leg syndrome, spasmodic torticollis, geniospasm, graft induced dyskinesia (a side effect that may develop after intrastriatally grafting embryonic mesencephalic tissue into the brains of patients with Parkinson's disease) or movement disorders associated therewith.

Movement disorders according to the present invention may also be associated with use of neuroleptic drugs, idiopathic disease, genetic dysfunctions, infections or other conditions which lead to dysfunction of the basal ganglia and/or lead to altered synaptic DA levels.

One embodiment of the present invention relates to treatment of symptoms of the movement disorders as defined herein, and of disorders or conditions associated with the movement disorders.

Parkinson's disease is associated with muscle rigidity, tremor, postural abnormalities, gait abnormalities, a slowing of physical movement (bradykinesia), and in extreme cases a loss of physical movement (akinesia). PD is caused by degeneration and death of dopaminergic neurons in substantia nigra pars compacta, and leads to dysfunctional regulation of dopamine neurotransmission.

In one particularly preferred embodiment of the present invention the movement disorder is Parkinson's disease. Another particularly preferred embodiment of the present invention is treatment of movement disorders associated with Parkinson's disease such as L-DOPA induced dyskinesia.

Administration of L-DOPA to unilaterally 6-OHDA-lesioned rats induces abnormal involuntary movements (AIMs) and changes in concentrations of neurotransmitters in the brain. Using methodologies known in the art such as fore example PET scanning it is possible to measure levels of such neurotransmitters (e.g. dopamine, gamma amino butyric acid (GABA), noradrenalin, serotonin) in different brain regions in freely moving rats that previously have been treated with 6-OHDA. This procedure allows for a direct comparison between central neurotransmitters and behavior and is a method used to determine mechanism of action and efficacy of compounds of the present invention.

In another embodiment of the present invention, the movement disorder is caused by or associated with medication of antipsychotics such as haloperidol, droperidol, pimozide, trifluoperazine, amisulpride, risperidone, aripiprazole, asenapine, and zuclopenthixol, antidepressants such as fluoxetine, paroxetine, venlafaxine, and trazodone, anti-emetic drugs such as dopamine blockers for example metoclopramide (reglan) and prochlorperazine (compazine).

In yet another embodiment of the present invention, the movement disorder is caused by or associated with withdrawal of opioids, barbiturates, cocaine, benzodiazepines, alcohol, or amphetamines.

With the use of PET scanning it is possible to measure levels of neurotransmitters which may be increased or decreased (e.g. dopamine, gamma amino butyric acid (GABA), noradrenalin, serotonin) in the brain region depending of the type of movement disorder. The dopamine levels and PET scanning procedures are useful to study levels of dopamine and dopamine receptors in healthy and disease animals and humans and thereby study effects of drug treatment in the diseases as described herein. Furthermore this procedure can be used to predict effects in humans from animal studies and are useful for predicting efficacy of drug combinations of the current invention. A commonly used PET tracer for studying dopamine levels in human volunteers, in patients suffering from Parkinson's disease and in animal models of Parkinson's disease is [¹¹C]-raclopride. Raclopride is a ligand for the dopamine D2 and D3 receptors. Using PET scanning, this tracer allows for a determination of changes in extracellular dopamine levels caused by treatment with drugs and drug combinations as described herein.

### Dosage and Dosing Regimes

The dosage requirements will vary with the particular drug composition employed, the route of administration and the particular subject being treated. It will also be recognized by one of skill in the art that the optimal quantity and spacing of individual dosages of a compound or a pharmaceutically acceptable derivative thereof will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of a compound or a pharmaceutically acceptable derivative thereof given per day for a defined number of days, can be ascertained using conventional course of treatment determination tests.

The KCNQ channel activators and 5-HT1 receptor agonists may be administered simultaneously, sequentially or separately in single doses or as several doses. Thus, the KCNQ channel activators and 5-HT1 receptor agonists and pharmaceutical compositions or kit of parts comprising both of these compounds may be administered one or several times per day, for example such as from 1 to 5 times per day, preferably such as 1 to 3 times per day. In other embodiments, the compounds may be administered 1 time a day, such as 2 times a day, for example 3 times a day, such as 4 times a day, for example 5 times a day, such as 6 times a day, for example 7 times a day, such as 8 times a day.

### Dosage

The term “unit dosage form” as used herein refers to physically discrete units suitable as unitary dosages for human and animal subjects, each unit containing a predetermined quantity of a compound, alone or in combination with other agents, calculated in an amount sufficient to produce the desired effect in association with a pharmaceutically acceptable diluent, carrier, or vehicle. The specifications for the unit dosage forms of the present invention depend on the particular compound or compounds employed and the effect to be achieved, as well as the pharmacodynamics associated with each compound in the host. The dose administered should be an “effective amount” or an amount necessary to achieve an “effective level” in the individual patient.

When the “effective level” is used as the preferred endpoint for dosing, the actual dose and schedule can vary, depending on inter-individual differences in pharmacokinetics, drug distribution, and metabolism. The “effective level” can be defined, for example, as the blood or tissue level desired in the patient that corresponds to a concentration of one or more compounds according to the invention.

The combined use of KCNQ channel activators and 5-HT1 receptor agonists of the present invention can induce combined, additive or synergistic effects, which may enable for a lowered dosage of 5-HT1 receptor agonist and/or KCNQ channel activator in the treatment of movement disorders. The lowered dosage scheme can result in a reduced risk of adverse effects of treatment with 5-HT1 receptor agonists, such as reducing the risk of development of serotonin syndrome. Further, the combined use of 5-HT1 receptor agonists of the present invention may increase the efficacy of the treatment, for instance by prolonging the positive effects of the treatment, and/or by increasing the positive effects of treatment compared to other treatments known in the art.

According to the present invention, KCNQ channel activators and 5-HT1 receptor agonists are administered to individuals in need of treatment in pharmaceutically effective amounts. A therapeutically effective amount of a compound according to the present invention is an amount sufficient to cure, prevent, reduce the risk of, alleviate or partially arrest the clinical manifestations of a given disease or movement disorder and its complications. The amount that is effective for a particular therapeutic purpose will depend on the severity and the sort of the movement disorder as well as on the weight and general state of the subject.

In one embodiment of the present invention, the KCNQ channel activator is administered in daily doses in the range of 0.5 mg/day to 2000 mg/day, such as 0.5 mg/day to 1500 mg/day, or such as 0.5 mg/day to 1200 mg/day, or such as 100 mg/day to 1200 mg/day, or such as 200 mg/day to 1200 mg/day, or such as 300 mg/day to 1200 mg/day, wherein doses of 0.5 mg/day to 1200 mg/day are preferred and doses of 100 mg/day to 1200 mg/day are even more preferred.

In one other embodiment of the present invention, the KCNQ channel activator is administered in daily starting doses which may be increased gradually during time to reach a daily full dose. According to the present invention, such a starting dose is in the range of 0.5 mg/day to 500 mg/day, such as 0.5 mg/day to 400 mg/day, such as 0.5 mg/day to 300 mg/day, such as 0.5 mg/day to 150 mg/day, such as 0.5 mg/day to 75 mg/day, such as 0.5 mg/day to 50 mg/day. A daily full dose which is used after the starting period is according to the present invention in the range of 0.5 mg/day to 2000 mg/day, such as 0.5 mg/day to 1500 mg/day, or such as 0.5 mg/day to 1200 mg/day, or such as 200 mg/day to 1200 mg/day, or such as 300 mg/day to 1200 mg/day, or such as 200 mg/day to 1200 mg/day, or such as 600 mg/day to 1200 mg/day.

Preferably a starting dose is in the range of 0.5 mg/day to 600 mg/day, and a daily full dose is in the range of 600 mg/day to 1200 mg/day.

In a preferred embodiment of the present invention, retigabine is administered in daily starting doses of 0.5 mg/day to 600 mg/day, and in daily full doses of 600 mg/day to 1200 mg/day.

In one embodiment of the present invention, the 5-HT1 receptor agonist is administered in doses of 0.5 mg/day to 100 mg/day, such as 0.5 mg/day to 1 mg/day, such as 1 mg/day to 5 mg/day, such as 1 mg/day to 2 mg/day, or such as 2 mg/day to 5 mg/day, or such as 5 mg/day to 10 mg/day, or such as 5 mg/day to 10 mg/day, or such as 10 mg/day to 20 mg/day, or such as 20 mg/day to 30 mg/day, or such as 30 mg/day to 40 mg/day, or such as 40 mg/day to 50 mg/day, or such as 40 mg/day to 60 mg/day, or such as 60 mg/day to 70 mg/day, or such as 70 mg/day to 80 mg/day, or such as 80 mg/day to 90 mg/day, or such as 90 mg/day to 95 mg/day, or such as 95 mg/day to 98 mg/day, or such as 98 mg/day to 100 mg/day, preferably in doses of 0.5 mg/day to 60 mg/day and even more preferably in doses of 0.5 mg/day to 30 mg/day, such as such as 0.5 to 5 mg/day, or such as 5 mg/day to 10 mg/day, or such as 10 mg/day to 15 mg/day, or such as 15 mg/day to 30 mg/day

In a preferred embodiment of the present invention, buspirone is administered in doses of 0.5 mg/day to 60 mg/day, more preferably in doses of 0.5 mg/day to 30 mg/day.

In one embodiment of the present invention the dose of a KCNQ channel activator, or a 5-HT1 receptor agonist, or a pharmaceutical composition according to the present invention is adjusted to the bodyweight of the treated individual. Such a dose can be in the range of 0.05 mg/kg bodyweight to 100 mg/kg bodyweight, such as in the range of 0.05 mg/kg bodyweight to 50 mg/kg bodyweight such as in the range of 0.05 mg/kg bodyweight to 30 mg/kg bodyweight, or such as in the range of 0.5 mg/kg bodyweight to 15 mg/kg bodyweight, or such as 5 mg/kg bodyweight to 10 mg/kg bodyweight.

### Dosage Regimens

In one embodiment of the present invention the KCNQ channel activator and the serotonin 5-HT1 receptor agonist are comprised within the same pharmaceutical composition.

In one embodiment of the present invention the KCNQ channel activator and the serotonin 5-HT1 receptor agonist are comprised in separate pharmaceutical compositions to provide a kit of parts.

When the KCNQ channel activator and the serotonin 5-HT1 receptor agonist are comprised within the same pharmaceutical composition, both compounds may in one embodiment be released or administered simultaneously. Alternatively, both compounds may in one embodiment be released or administered sequentially.

When the KCNQ channel activator and the serotonin 5-HT1 receptor agonist are comprised in separate pharmaceutical compositions to provide o kit of parts, both compounds may in one embodiment be released or administered simultaneously. Alternatively, both compounds may in one embodiment be released or administered sequentially.

In one embodiment wherein the KCNQ channel activator and the serotonin 5-HT1 receptor agonist are released or administered sequentially, the KCNQ channel activator is released or administered before the serotonin 5-HT1 receptor agonist; or the KCNQ channel activator is released or administered before and during release or administration of the serotonin 5-HT1 receptor agonist.

In one embodiment wherein the KCNQ channel activator and the serotonin 5-HT1 receptor agonist are released or administered sequentially, the serotonin 5-HT1 receptor agonist is released or administered before the KCNQ channel activator; or wherein the serotonin 5-HT1 receptor agonist is released or administered before and during release or administration of the KCNQ channel activator.

In a particular embodiment, the one compound is released or administered between 5 minutes and 240 minutes before the other compound, such as between 5 and 15 minutes, for example between 15 and 30 minutes, such as between 30 minutes and 60 minutes, such as between 60 and 90 minutes, such as between 90 and 120 minutes, such as between 120 and 180 minutes, such as between 180 and 240 minutes.

### Other Active Ingredients

The compounds or pharmaceutical compositions of the present invention may be combined with or comprise one or more other active ingredients which are understood as other therapeutic compounds or pharmaceutically acceptable derivatives thereof.

Another active ingredient according to the present invention may further be one or more agents selected from the group of agents increasing the dopamine concentration in the synaptic cleft, dopamine, L-DOPA or dopamine receptor agonists or derivatives thereof. Thus, according to the present invention second active ingredients comprise DA receptor agonists, such as bromocriptine, pergolide, pramipexole, ropinirole, piribedil, cabergoline, apomorphine, lisuride, and derivatives thereof.

Other active ingredients may further be selected from the group of compounds which ameliorate PD symptoms or which are used for treatment of PD, such as peripheral inhibitors of the transformation of L-DOPA or (other dopamine prodrugs) to dopamine, for example carboxylase inhibitors such as carbidopa or benserazide, or NMDA antagonists such as for example amatidine (Symmetrel), catechol-O-methyl transferase (COMT) inhibitors such as for example tolcapone and entacapone, MAO-B inhibitors such as for example selegiline and rasagiline, serotonin receptor modulators, kappa opioid receptors agonists such as for example TRK-820 ((E)-N-[17-cyclopropylmethyl)-4,5α-epoxy-3,14-dihydroxymorphinan-6β-yl]-3-(furan-3-yl)-N-methylprop-2-enamide monohydrochloride), GABA modulators, modulators of neuronal potassium channels such as flupirtine and retigabine, and glutamate receptor modulators.

In a preferred embodiment of the present invention, another active ingredient is a dopamine prodrug, such as L-DOPA or a pharmaceutically acceptable derivative thereof.

In one embodiment of the present invention, the compounds or pharmaceutical compositions may be combined with two or more other active ingredients. Such two other active ingredients may be L-DOPA in combination with a carboxylase inhibitor. Thus in an embodiment of the present invention, the two or more other active ingredients comprise L-DOPA and carbidopa, or L-DOPA and benserazide.

In another embodiment, such two other active ingredients are L-DOPA in combination with a COMT inhibitor, wherein the COMT inhibitor can be tolcapone, or entacapone.

The other active ingredients according to the present invention can also be included in the same formulations such as for example the L-DOPA/benserazide formulations sinemet, parcopa, madopar, or L-DOPA/COMT inhibitor formulations such as for example stalevo.

### Routes of Administration

### Systemic Treatment

The main routes of administration are oral and parenteral in order to introduce the KCNQ channel activators and 5-HT1 receptor agonists into the blood stream to ultimately target the sites of desired action (i.e. in the neural system, such as the brain). Appropriate dosage forms for such administration may be prepared by conventional techniques.

### Oral Administration

Oral administration is normally for enteral drug delivery, wherein the KCNQ channel activators or the 5-HT1 receptor agonists or both are delivered through the enteral mucosa. In a preferred embodiment of the present invention, the KCNQ channel activator, or the 5-HT1 receptor agonist, or a pharmaceutical composition as defined herein are orally administered.

### Parenteral Administration

It will be appreciated that the preferred route will depend on the general condition and age of the subject to be treated, the nature of the condition to be treated.

Parenteral administration is any administration route not being the oral/enteral route whereby the medicament avoids first-pass degradation in the liver. Accordingly, parenteral administration includes any injections and infusions, for example bolus injection or continuous infusion, such as intravenous administration, intramuscular administration and subcutaneous administration. Furthermore, parenteral administration includes inhalations and topical administration.

Accordingly, the KCNQ channel activator, and/or the 5-HT1 receptor agonist, or a pharmaceutical composition as defined herein, may be administered topically to cross any mucosal membrane of an animal to which the biologically active substance is to be given, e.g. in the nose, vagina, eye, mouth, genital tract, lungs, gastrointestinal tract, or rectum, preferably the mucosa of the nose, or mouth, and accordingly, parenteral administration may also include buccal, sublingual, nasal, rectal, vaginal and intraperitoneal administration as well as pulmonal and bronchial administration by inhalation or installation. Also, the agent may be administered topically to cross the skin.

Of parenteral administration forms, the subcutaneous and intramuscular forms of parenteral administration are generally preferred.

### Pharmaceutically Acceptable Derivatives

Pharmaceutically acceptable salts of the instant compounds, where they can be prepared, are also intended to be covered by this invention. These salts will be ones which are acceptable in their application to a pharmaceutical use. By that it is meant that the salt will retain the biological activity of the parent compound and the salt will not have untoward or deleterious effects in its application and use in treating diseases.

The salts of the present invention, such as pharmaceutically acceptable salts, e.g. pharmaceutically acceptable acid addition salts, refers to the relatively non-toxic, inorganic and organic addition salts of compounds of the present invention. These salts can be prepared in situ during the final isolation and purification of the compounds or by separately reacting the purified compound in its free acid or base form with a suitable organic or inorganic compound and isolating the salt thus formed. The compounds of the present invention are capable of forming a wide variety of different salts with various inorganic and organic acids. Although such salts must be pharmaceutically acceptable for administration to animals, it is often desirable in practice to initially isolate the base compound from the reaction mixture as a pharmaceutically unacceptable salt and then simply convert to the free base compound by treatment with an alkaline reagent and thereafter convert the free base to a pharmaceutically acceptable acid addition salt.

The pharmaceutically acceptable acid addition salts of the compounds of the present invention are prepared by contacting the compounds with a sufficient amount of the desired acid to produce the salt in the conventional manner. The compounds as such may be regenerated by contacting the salt form with a base and isolating it in a conventional manner. The compounds as such differ from their respective salt forms somewhat in certain physical properties such as solubility in polar solvents, but otherwise the salts are equivalent to their respective compounds for purposes of the present invention.

Salts may e.g. be prepared from inorganic acids comprising sulfate, pyrosulfate, bisulfate, sulfite, bisulfite, nitrate, phosphate, monohydrogenphosphate, dihydrogenphosphate, metaphosphate, pyrophosphate, chloride, bromide, iodide such as hydrochloric, nitric, phosphoric, sulfuric, hydrobromic, hydriodic, phosphorus, trifluoromethanesulfonate, and the like.

Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like.

Salts may also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, such as carbonic formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene-salicylic, ethanedisulfonic, gluconic, citraconic, aspartic, stearic, palmitic, EDTA, glycolic, p-aminobenzoic, glutamic, benzenesulfonic and p-toluenesulfonic, pbromophenyl-sulfonic acid, and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, trifluoromethanesulfonate and the like. (See, for example, Berge S. M. et al., “Pharmaceutical Salts,” J. Pharm. 1977; 66:1-19 which is incorporated herein by reference.)

Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenyl butyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Preferred acid addition salts are those formed with mineral acids such as hydrochloric acid and hydrobromic acid, and, especially, hydrochloric acid. An example of such a salt is for example buspirone hydrochloride.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

Examples of metal salts include lithium, sodium, potassium and magnesium salts, and the like. Examples of ammonium and alkylated ammonium salts include ammonium, methylammonium, dimethylammonium, trimethylammonium, ethylammonium, hydroxyethylammonium, diethylammonium, butylammonium and tetramethylammonium salts, and the like.

Further examples of pharmaceutically acceptable inorganic or organic acid addition salts include the pharmaceutically acceptable salts listed in J. Pharm. Sci. 1977, 66, 2, the contents of which are incorporated herein by reference.

In one embodiment of the present invention, the KCNQ channel activator and/or the 5-HT1a agonist is on crystalline forms, for example co-crystallized forms or hydrates of crystalline forms.

In a one specific embodiment of the invention when the KCNQ channel activator is flupritine, a pharmaceutically acceptable salt is a maleate salt of flupirtine, which may or may not be a co-crystal as described in the art such as for example in EP2206699 and EP 2206700.

Pharmaceutically active derivatives of KNQC channel activators can for instance be salts as described in US 2007191351 or for example 1,4 diamino bicyclic retigabine analogues as described in WO 2008066900.

The combinations of compounds which are KCNQ channel activators or 5-HT1 receptor agonists according to the present invention preferably activate ion channels and receptors in the neuronal system, such as the brain. Therefore, in one embodiment of the present invention, the pharmaceutical derivatives of the compounds or pharmaceutical compositions as defined herein enable the KCNQ channel activator and/or the 5-HT1 receptor agonist to cross the blood-brain barrier.

The term “prodrug” refers to compounds that are rapidly transformed in vivo to yield the parent compound of the above formulae, for example, by hydrolysis in blood or by metabolism in cells, such as for example the cells of the basal ganglia. A thorough discussion is provided in T. Higuchi and V Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference. Examples of prodrugs include pharmaceutically acceptable, non-toxic esters of the compounds of the present invention. Esters of the compounds of the present invention may be prepared according to conventional methods “March's Advanced Organic Chemistry, 5^{th} Edition”. M. B. Smith & J. March, John Wiley & Sons, 2001.

### Pharmaceutical Formulations

The present invention relates to compounds which are KCNQ channel activators or 5-HT1 receptor agonists and to pharmacological compositions or kit of parts comprising both a KCNQ channel activators and a 5-HT1 receptor agonist.

The compounds and pharmaceutical compositions or kit of parts according to the invention may be administered with at least one other active compound.

The compounds or pharmacological compositions may be administered simultaneously, either as separate formulations or combined in a unit dosage form, or administered sequentially.

The combinations of compounds or pharmacological compositions according to the invention may be included in a kit of parts comprising the compounds or pharmaceutical compositions of the invention for simultaneous, sequential or separate administration.

Whilst it is possible for the compounds of the present invention to be administered as the raw chemical or as a pharmaceutically acceptable derivative such as a salt thereof, it is preferred to present them in the form of a pharmaceutical formulation. Accordingly, the present invention further provides a pharmaceutical formulation which comprises a compound of the present invention, or a pharmaceutically acceptable derivative such as a salt or ester thereof, and a pharmaceutically acceptable carrier therefore. The pharmaceutical formulations may be prepared by conventional techniques, e.g. as described in Remington: The Science and Practice of Pharmacy 2005, Lippincott, Williams & Wilkins.

The pharmaceutically acceptable carriers can be either solid or liquid. Solid form preparations include powders, tablets, pills, capsules, cachets, suppositories, and dispersible granules. A solid carrier can be one or more excipients which may also act as diluents, flavoring agents, solubilizers, lubricants, suspending agents, binders, preservatives, wetting agents, tablet disintegrating agents, or an encapsulating material.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The compounds and pharmaceutical compositions of the present invention may be formulated for parenteral administration and may be presented in unit dose form in ampoules, pre-filled syringes, small volume infusion or in multi-dose containers, optionally with an added preservative. The compositions may take such forms as suspensions, solutions, or emulsions in oily or aqueous vehicles, for example solutions in aqueous polyethylene glycol. Examples of oily or non-aqueous carriers, diluents, solvents or vehicles include propylene glycol, polyethylene glycol, vegetable oils (e.g., olive oil), and injectable organic esters (e.g., ethyl oleate), and may contain agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compounds of the invention may also be formulated for topical delivery. The topical formulation may include a pharmaceutically acceptable carrier adapted for topical administration. Thus, the composition may take the form of a suspension, solution, ointment, lotion, sexual lubricant, cream, foam, aerosol, spray, suppository, implant, inhalant, tablet, capsule, dry powder, syrup, balm or lozenge, for example.

Preferably, the formulation will comprise about 0.5% to 75% by weight of the active ingredient(s) with the remainder consisting of suitable pharmaceutical excipients as described herein.

Sustained or controlled release formulations of the KCNQ channel activators, 5-HT1 agonists, and pharmaceutical compositions of the present invention are also within the scope of the present invention. Such formulations include for example a sustained release formulation comprising retigabine as described in WO 02/80898 and WO01/66081, or for example controlled release formulations of buspirone for example as described in U.S. Pat. No. 5,431,922; EP 1266656; U.S. Pat. No. 5,633,009.

### Combined Oral Formulations

In a particular embodiment the pharmaceutical composition comprising a KCNQ channel activator and a serotonin 5-HT1 receptor agonist according to the present invention are combined in an oral formulation that will release the KCNQ channel activator and the serotonin 5-HT1 receptor agonist at the same time or sequentially.

Time release technology (extended or sustained release) is a mechanism used in pill tablets or capsules to dissolve slowly and release a drug over time. The advantages of extended-release tablets or capsules are that they may be taken less frequently than immediate-release formulations, and that they keep steadier levels of the drug in the bloodstream. Another advantage is that the drug release profiles for each of the two or more constituents may differ to optimise the overall combination effect of such two or more drugs.

Time-release drugs may be formulated so that the active ingredient is embedded in a matrix of insoluble substance(s) such that the dissolving drug must find its way out through the holes in the matrix. Some drugs are enclosed in polymer-based tablets with a laser-drilled hole on one side and a porous membrane on the other side. Stomach acids push through the porous membrane, thereby pushing the drug out through the laser-drilled hole. In time, the entire drug dose releases into the system while the polymer container remains intact, to be excreted later through normal digestion. In some formulations, the drug dissolves into the matrix, and the matrix physically swells to form a gel, allowing the drug to exit through the gel's outer surface. Micro-encapsulation also produces complex dissolution profiles; through coating an active pharmaceutical ingredient around an inert core, and layering it with insoluble substances to form a microsphere a more consistent and replicable dissolution rate is obtained—in a convenient format that may be mixed with other instant release pharmaceutical ingredients, e.g. into any two piece gelatin capsule.

In one embodiment, the KCNQ channel activator and the serotonin 5-HT1 receptor agonist are both released by sustained release, and in another embodiment both compounds are released by immediate release.

In a particular embodiment, the KCNQ channel activator and the serotonin 5-HT1 receptor agonist are combined in a formulation such as an oral formulation (tablet, capsule etc.) in such a way (by such a formulation) that will release the one (first) compound before the other (second) compound and therefore will allow the compound first released to be absorbed and enter systemic circulation before the second released compound. This will allow the first compound to be absorbed and reach its target to induce the relevant changes before provision of the second compound.

In a particular embodiment, one compound is released from the composition by an extended release procedure, and the other compound is released from the composition by an immediate release procedure.

In a particular embodiment such formulation or tablet/capsule is designed to slowly release the one (first) compound by an extended (or delayed) release procedure, preferably before and/or during immediate release of the other (second) compound.

Alternatively, such formulation or tablet/capsule is designed to immediately release the one (first) compound by an immediate release procedure, preferably before and/or during extended release of the other (second) compound.

In a particular embodiment, one compound is released by an immediate release procedure. The immediate release procedure of the one first compound can mimic a bolus administration i.e. the administration of a substance in the form of a single, large dose. This will provide a peak dose of the compound.

In a particular embodiment of the invention a combination formulation as described herein above may be administered once or more, such as over an extended time period. In one embodiment, said formulation may be administered once per day, such as twice per day, for example 3 times per day, such as 4 times per day, for example 5 times per day, such as 6 times per day.

In one embodiment, said formulation may be administered daily (once or more per day) or intermittently with intervals of 1, 2, 3, 4, 5, 6 or 7 days, for a limited or an extended period of time, i.e. the treatment may be chronic from the onset of diagnosis.

The extended release formulation will provide a steady state concentration of the compound that provides for a lower total accumulated dose of the compound and a prolonged exposure as compared to immediate release. A lower dose will reduce adverse effects of the drug, and as such the formulation will be efficacious in treatment of movement disorders such as L-DOPA induced dyskinesia with rediced adverse effects.

In another embodiment the KCNQ channel activator and the serotonin 5-HT1 receptor agonist are combined in a formulation together with a second active ingredient. Such second active ingredient could be L-DOPA (or other dopamine pro-drugs) in combination with peripheral inhibitors of the transformation of L-DOPA (or other dopamine pro-drugs) to dopamine, for example L-DOPA decarboxylase inhibitors such as carbidopa or benserazide.

In a preferred embodiment such formulation is designed to release the KCNQ channel activator and the serotonin 5-HT1 receptor agonist, each at the same time or sequentially, at the same time or before the second active ingredient is released.

### Methods of Treatment

One aspect of the present invention relates to methods for treatment, prevention or alleviation of movement disorders. Such methods comprise either
a) one or more steps of administration of an effective amount of a pharmaceutical composition or a kit of parts as described herein, or
b) one or more steps of administration of an effective amount of a KCNQ channel activator and one or more steps of administration of an effective amount of a 5-HT1 receptor agonist as described herein, or
c) one or more steps of administration according to both a) and b) as defined above,
to an individual in need thereof.

In one embodiment of the present invention, a method for treatment further comprises one or more steps wherein increasing doses of a KCNQ channel activator is administered, such as one or more steps of administration of starting doses as described herein, and one or more steps of administration of a full daily dose as described herein.

In one embodiment of the present invention, a method of treatment as defined herein may further comprise simultaneous, sequential or separate administration of another active compound as described herein, such as for example an agent increasing the dopamine concentration in the synaptic cleft, dopamine, L-DOPA, dopamine receptor agonists or a pharmaceutically acceptable derivative thereof.

The positive effects of the use of a method according to the present invention can be assessed by using the conventional scales for measuring the degree of movement disorders, such as the Lang-Fahn Activities of Daily Living Dyskinesia scale, Clinical Global Impression, Unified Parkinson's Disease Rating Scales as well as the Abnormal Involuntary Movement Scale (AIMS) and Barnes Akathisia Scale (BAS).

### Kit of Parts

One aspect of the present invention relates to a kit of parts comprising the combination of compounds as defined herein. Thus in one embodiment of the present invention a kit of parts is provided which comprises:
a) a KCNQ channel activator as defined herein and a 5-HT1 receptor agonist as defined herein, and/or
b) a pharmaceutical composition as defined by the present invention,
   for simultaneous, sequential or separate administration.

In one embodiment of the present invention, said kit of parts is used for treatment, prevention or alleviation of movement disorders.

Said kit of parts may further comprise one or more other active ingredients for simultaneous, sequential or separate administration, such as an agent increasing the dopamine concentration in the synaptic cleft, dopamine, L-DOPA, dopamine receptor agonists or a pharmaceutically acceptable derivative thereof.

### REFERENCES

Bonifati et al., Clin NeurPharmacol, 1994, 17, 73-82.
Dekundy et al: Behavioural Brain Research 179 (2007) 76-89
Del Sorbo and Albanese: J. Neurol. 2008; 255 Suppl 4: 32-41.
Elangbam et al: J Histochem Cytochem 53:671-677, 2005
Filip et al. Pharmacol. Reports. (2009) 61, 761-777; Ohno, Central Nervous System Agents in Medicinal Chemistry, 2010, 10, 148-157.
Fox et al: Movement Disorders Vol. 24, No. 9, 2009.
Grégoire et al: Parkinsonism Relat Disord. 2009; 15(6): 445-52.
Jenner: Nat Rev Neurosci. 2008; 9(9): 665-77.
Jentsch Nature Reviews Neuroscience 2000, 1, 21-30.
Kirk et al.: J. Neurosci 2001; 21:2889-96
Moss et al: J Clin Psychopharmacol. 1993 June; 13(3):204-9.
Muñoz et al: Brain. 2008; 131(Pt 12): 3380-94
Muñoz et al: Experimental Neurology 219 (2009) 298-307.
Newman-Tancredi: Current Opinion in Investigational Drugs 2010 11(7):802-812.
Ohno, Central Nervous System Agents in Medicinal Chemistry, 2010, 10, 148-157
Remington: The Science and Practice of Pharmacy, 20^{th} Edition, Gennaro, Ed., Mack Publishing Co., Easton, Pa., 2000
Roppongi et al: Prog Neuropsychopharmacol Biol Psychiatry. 2007; 31(1):308-10.
Schallert et al., J. Neural Transpl Plast 1992; 3:332-3
Wulff et al., Nat Rev Drug Discov. 2009 December; 8(12):982-1001.
Xiong Q, Gao Z, Wang W, Li M. Trends Pharmacol Sci. 2008 February; 29(2):99-107.

### EXAMPLES

### Example I

### Determination of Activation of the Serotonin 5-HT1 Receptors

The [³⁵S]-GTPγS assay can be used to determine the ability of a compound to activate one or more cloned receptors of the serotonin 5-HT1 receptor family and thus act as a 5-HT1 receptor agonist. When using such assays, a selective agonist would be an agonist which only activates one type of 5-HT1 receptor, whereas no or no significant activity is observed with other types of expressed 5-HT1 receptor. A combined agonist which activates several 5-HT1 receptors would in the same type of assay activate several different expressed 5-HT1 receptors.

### Membrane Preparation

Assays are performed with cells expressing one or more of the cloned human 5-HT1A, 5-HT1B, 5-HT1D and 5-HT1F receptors. On the assay day, an aliquot of cells (stored at −70° C.) is thawed and re-suspended in 50 mM Tris-HCl, pH 7.4, and centrifuged at 39,800 g for 10 min at 4° C. The resulting pellet is re-suspended in 50 mM Tris-HCl, pH 7.4, incubated for 10 min at 37° C., and centrifuged at 39,800 g for 10 min at 4° C. The pellet is re-suspended and centrifuged once more, with the final pellet being suspended in 4 mM MgCl2, 160 mM NaCl, 0.267 mM EGTA, 67 mM Tris-HCl, pH 7.4 for the [³⁵S]-GTPγS binding assays.

### Binding Assay

The method for the 5-HT1 receptor [35S]-GTPγS binding assays are, adapted to an SPA (scintillation proximity assay) format. Incubations are performed in a total volume of 200 ml in 96-well assay plates. [³⁵S]-GTPγS and guanosine-50-diphosphate (GDP) in assay buffer (MgCl2, NaCl, EGTA in Tris-HCl, pH 7.4; 50 ml) is added to 50 ml of test compounds diluted in water. WGA (wheat germ agglutinin) beads (Amersham Pharmacia Biotech Inc., Piscataway, N.J., USA) for SPA in assay buffer (50 ml) are then added. Membrane homogenate (50 ml) from cells expressing the cloned human 5-HT1A receptor in assay buffer is added, and the plates are covered with sealing tape (PerkinElmer Wallac, Inc., Gaithersburg, Md., USA) and allowed to incubate at room temperature for 2 h.

The final concentrations of MgCl₂, NaCl, EGTA, GDP, [³⁵S]-GTPγS, and Tris are 3 mM, 120 mM, 0.2 mM, 10 mM, approximately 0.3 nM, and 50 mM, respectively. The plates are then centrifuged at approximately 200×g for 10 min at room temperature. The amount of [³⁵S]-GTPγS bound to the membranes, i.e. in close proximity to the WGA SPA beads, is then determined using a Wallac MicroBeta® Trilux Scintillation Counter (PerkinElmer Wallac, Inc.).

### Data Analysis

Using GraphPad Prism software, non-linear regression analysis is performed on the concentration-response curves (generating EC₅₀ and Emax values for stimulation of [³⁵S]-GTPγS binding) using a four-parameter logistic equation. Efficacy (Emax) values, determined by the non-linear regression analysis, for the selected compounds, is expressed as the percentage of [³⁵S]-GTPγS binding relative to the response produced by 10 mM of agonists for the 5-HT1A, 5-HT1B, 5-HT1E or 5-HT1F receptors or 1 mM 5-HT agonist for the 5-HT1D receptor which is run as a standard with each concentration-response curve.

### Example II

### Determination of Activation of KCNQ Channels

The KCNQ (Kᵥ7) channels in the brain belong to the family of voltage-dependent potassium channels. Four subunits termed KCNQ2-5 have been identified that form both homo- and heteromeric complexes. KCNQ channel openers, such as retigabine, increase the opening probability of the channels by shifting the voltage-dependency to more negative voltages.

Expression in Xenopus laevis Oocytes

Female Xenopus laevis are anaesthetized by immersion in a 0.4% (w/v) solution of 3-aminobenzoic acid ethyl ester (Sigma, St. Louis, Mo., USA) for 15-20 min. Ovarian lobes are cut off through a small abdominal incision and subsequently defolliculated by enzymatic treatment with 0.5 mg/mL collagenase type IA (Sigma, St. Louis, Mo., USA) in OR2 solution (in mM: 82.5 NaCl, 2 KCl, 1 MgCl₂, 5 HEPES, pH 7.4) for 3 hours. Oocytes are then kept in Modified Barth's Saline (in mM: 88 NaCl, 1 KCl, 2.4 NaHCO₃, 0.41 CaCl₂, 0.82 MgSO₄, 0.3 Ca(NO₃)₂, 15 HEPES, pH 7.4 suppl. with 100 U/mL penicillin and 100 μg/mL streptomycin) at 18° C. until injection. cRNA was injected using a Nanoliter Injector (World Precision Instruments, Sarasota, Fla., USA). For Kv7.1 between 2 and 10 ng of cRNA is injected, for Kv7.2 and Kv7.5 10-25 ng is injected and for Kv7.4 3-6 ng is injected. For co-expression of Kv7.2 and Kv7.3 2 ng of each is injected. The oocytes are kept in Modified Barth's Saline at 18° C. and currents were recorded after 2-7 days.

### Characterisation of KCNQ Channel Currents Using Electrophysiology in X. Laevis Oocytes

KCNQ currents in Xenopus laevis oocytes can be recorded using two-electrode voltage-clamp. These recordings are performed at room temperature in Ringer buffer (in mM:115 NaCl, 2.5 KCl, 1.8 CaCl2, 0.1 MgCl2, 10 HEPES, pH 7.4) using an Axon GeneClamp 500B two-electrode voltage-clamp amplifier (Axon Instruments Inc., Union City, Calif., USA) and a Digidata 1440A digitizer (Axon Instruments) (Blom et al., PLoS One 2009, 4:e8251). The oocytes are placed in a perfusion system connected to a continuous flow system, and effects of KCNQ channel activators (also called KCNQ positive modulators) are determined in increasing concentrations. Electrodes are pulled from filamented borosilicate glass capillaries and filled with 1 M KCl. The electrodes have a resistance of 0.5-2.5 MV.

DA Release from Minced Striatal Slices

Another method to determine neuronal effects of KCNQ openers in vitro is the dopamine release assays in striatal slices according to previously published methods (Jensen et al (2011) Basic Clin Pharmacol Toxicol. 2011 May 21. doi: 10.1111/j.1742-7843.2011.00730.x. Striatal tissue dissected from was chopped at 150 μm using a tissue chopper (Brinkman Instruments, Westbury, N.Y.) three times with a 60 degree rotation in between. The slices were resuspended in 37° C. Krebs buffer (KB; 118 mM NaCl, 2.4 mM KCl, 2.4 mM CaCl₂, 1.2 mM MgSO₄, 1.2 KH₂PO₄, 25 mM NaHCO₃, 10 mM D-glucose, oxygenated with 95% O₂/5% CO₂ for 1 h, pH 7.4) and triturated 5 times to further dissociate the tissue. Slices were washed in KB and incubated with 50 nM [³H]-dopamine (specific activity 38.7 Ci/mmol, Perkin Elmer, Waltham, USA) at 37° C. for 30 min with 1 mM ascorbic acid and 10 μM pargyline. After one wash with KB containing 1 μM nomifensine, the slices were distributed in a 96-well filter-bottom microplate (Multiscreen® HTS, Millipore, Billerica, Mass.). Slices were resuspended in KB and incubated at 37° C. for 10 min and the filtrate was collected for determination of basal release. KB containing 16 mM KCl and/or increasing concentrations of positive KCNQ modulator was then added and incubated for 5 min at 37° C. and the filtrate was subsequently collected for determination of stimulated release. Finally, the cells were lysed by incubation in 0.1 M HCl for 1 h at 37° C. and the filtrate was collected. The radioactivity was determined by counting in a Topcount NXT™ microplate scintillation counter.

### Data Analysis

The data were analyzed by calculating the fractional release of [³H]-DA as the amount of radioactivity released during the stimulation period relative to the total radioactivity present before stimulation. The basal release was subtracted to give the evoked fractional release, which was normalized to the response elicited by 16 mM KCl. IC₅₀ values for concentration-inhibition curves were calculated using non-linear regression analysis in GraphPad Prism 5 software.

### Example III

### Evaluation of the 5-HT1 Receptor Agonist Buspirone and the KCNQ Positive Modulator Retigabine for Treatment of Movement Disorders Associated with Parkinson's Disease and LID

The present study describes the evaluation of buspirone and retigabine in the 6-OHDA rat model. 6-OHDA (6-hydroxydopamine) is a neurotoxin that selectively kills dopaminergic and noradrenergic neurons and induces a reduction of dopamine levels in the brain. Administration of L-DOPA to unilaterally 6-OHDA-lesioned rats induces abnormal involuntary movements (AIMs). These are axial, limb and oral movements that occur only on the body side that is ipsilateral to the lesion. AIM rat models have been shown useful because they respond to a number of drugs which have been shown to suppress dyskinesia (including PD) in humans.

### Animals:

60 Sprague-Dawley (SD) male rat (bred in house, originally from SLAC Laboratory Animal Co. Ltd) at 9-week of age at body weight of 200 to 250 g from Shanghai SLAC Co. Ltd. arrived at the laboratory at least 1 week prior to behavioural testing. Rats were housed in groups of n=2/cage. Animals had ad libitum access to standard rodent chow and water. Animal housing and testing rooms were maintained under controlled environmental conditions and were within close proximity of each other. Animal housing rooms were on a 12-hour light-dark cycle with lights on at 6:00 AM and maintained at 70° F./21° C. (range: 68-72° F./20-22° C.) with a humidity range of 20-40%. Testing rooms were maintained at 68-72° F. with a humidity range of 20-40%.

### 6-OHDA Lesion Surgery:

Dopamine (DA)-denervating lesions were performed by unilateral injection of 6-OHDA in the ascending nigrostriatal pathway. Rats were anesthetized with pentobarbital sodium 40 mg/kg (i.p.—intraperitoneal injection) and positioned in a stereotactic frame.

6-OHDA was injected into the right ascending DA bundle at the following coordinates (in mm) relative to bregma and dural surface: (1) toothbar position −2.3, A=−4.4, L=1.2, V=7.8, (7.5 ug 6-OHDA), (2) toothbar position +3.4, A=−4.0, L=0.8, V=8.0 mm (6 ug 6-OHDA). Alternatively only one injection was made with the following coordinates: Tooth bar: −3.3 mm, AP: −1.8 mm, ML: −2.0 mm, DV: −8.6 mm (18 μg/6 μl 6-OHDA). The neurotoxin injections were performed at a rate of 1 ul/min, and the injection cannula was left in place for an additional 2-3 min thereafter.

After recovery from surgery, rats with nearly complete (>90%) lesions were selected by means of an apomorphin-induced rotation test. Intraperitoneal (i.p.) injection of 0.5 mg/kg apomorphine.HCl (Sigma) in saline evoked contralateral turning, which is considered to be the result of denervated hypersensitivity of DA receptors in the lesion side. Rotational behaviour in response to DA agonists grossly correlates with the severity of the lesion. Quantification of the rotational response was accomplished in rats by counting the turns in 30 minutes. Rats with rotational score 6 turns/min were selected for next tests. Animals were then allocated into two well-matched sub-groups (according to the amphetamine rotation) and received daily treatment as described below.

### Drugs and Treatment Regimens:

L-DOPA methyl ester (Sigma, Cat No. D9628Lot. No. 030M1604V)) was given at the dose of 6 mg/kg/day, combined with 15 mg/kg/day of benserazide.HCl. Chronic treatment with this dose of L-DOPA and benserazide was given for 3 weeks or more to all the rats with good lesions in order to induce a gradual development of dyskinesia-like movements. Thereafter, rats that had not developed dyskinesia were excluded from the study, and the rats with a cumulative AIM score ≧28 points over five testing sessions (dyskinesia severity grade≧2 on each axial, limb and orolingual scores) were kept on a drug treatment regimen of at least two injections of L-DOPA/benserazide per week in order to maintain stable AIM scores. The selected rats were allocated groups of 9-12 animals each, which were balanced with the respect to AIM severity. The animals were then treated with the drug and drug combinations as described below.

### L-DOPA Induced AIMs and Drugs Screening Test

AIMs ratings was performed by an investigator who was kept unaware of the pharmacological treatment administered to each rat (experimentally blinded). In order to quantify the severity of the AIMs, rats were observed individually in their standard cages every 20th minute at 20-180 min after an injection of I-DOPA. The AIM's were classified into four subtypes:

(A) axial AIMs (‘Ax’), i.e., dystonic or choreiform torsion of the trunk and neck towards the side contralateral to the lesion. In the mild cases: lateral flexion of the neck or torsional movements of the upper trunk towards the side contralateral to the lesion. With repeated injection of L-DOPA, this movement may develop into a pronounced and continuous dystonia-like axial torsion.
(B) limb AIMs (‘Li’), i.e. jerky and/or dystonic movements of the forelimb contralateral to the lesion. In mild cases: hyperkinetic, jerky stepping movements of the forelimb contralateral to the lesion, or small circular movements of the forelimb to and from the snout. As the severity of dyskinesia increases (which usually occurs with repeated administration of L-DOPA), the abnormal movements increase in amplitude, and assume mixed dystonic and hyperkinetic features. Dystonic movements are caused by sustained co-contraction of agonist/antagonist muscles; they are slow and force a body segment into unnatural positions. Hyperkinetic movements are fast and irregular in speed and direction. Sometimes the forelimb does not show jerky movements but becomes engaged in a continuous dystonic posture, which is also scored according to the time during which it is expressed.
(C) orolingual AIMs (‘OI’), i.e., twitching of orofacial muscles, and bursts of empty masticatory movements with protrusion of the tongue towards the side contralateral to the lesion. This form of dyskinesia affects facial, tongue, and masticatory muscles. It is recognizable as bursts of empty masticatory movements, accompanied to a variable degree by jaw opening, lateral translocations of the jaw, twitching of facial muscles, and protrusion of the tongue towards the side contralateral to the lesion. At its extreme severity, this subtype of dyskinesia engages all the above muscle groups with notable strength, and may also become complicated by self-mutilative biting on the skin of the forelimb contralateral to the lesion (easily recognizable by the fact that a round spot of skin becomes devoid of fur.
(D) locomotive AIMs (‘Lo’), i.e., increased locomotion with contralateral side bias. The latter AIM subtype was recorded in conformity with the original description of the rat AIM scale, although it was later established that locomotive AIMs do not provide a specific measure of dyskinesia, but rather provide a correlate of contralateral turning behavior in rodents with unilateral6-OHDA lesions.

Each of the four subtypes are scored on a severity scale from 0 to 4, where 0=absent, 1=present during less than half of the observation time, 2=present for more than half of the observation time, 3=present all the time but suppressible by external stimuli, and 4=present all the time and not suppressible by external stimuli.

The sum of locomotive, axial, limb, and orolingual AIM or axial, limb, and orolingual AIM scores per testing session were used for statistical analyses.

To determine the effects of specific doses of a combination of buspirone and retigabine the following group setting was used:

Vehicle: (saline, i.p., 30 min before L-DOPA, n=6)
Buspirone (0.5 mg/kg, i.p., n=6)
Retigabine (5 mg/kg, i.p. n=6)
Retigabine (1 mg/kg, i.p.)+Buspirone (0.5 mg/kg, i.p., n=6)
Retigabine (5 mg/kg, i.p.)+Buspirone (0.5 mg/kg, i.p., n=6)
Retigabine was given 35 minutes before L-DOPA while buspirone was given 30 minutes before L-DOPA.

The scores of Lo, Li, Ax, OL, were recorded every 20 minutes during a 2 h observation period for time course analysis.

The resulting AIM scores calculated as the sum of each of the subtypes locomotive, axial, limb, and orolingual AIM scores per testing session as well as total AIMs (Lo+Li+Ax+OL) per testing session were used for statistical analysis. Area under the curves (AUC) obtained from the above mentioned plot for each of the curves. The Area Under the Curves (AUCs) of total AIMs were calculated respectively according to the formula: ((Score₂₀ₘᵢₙ+Score₆₀ₘᵢₙ)/2+Score₄₀ₘᵢₙ)×20.

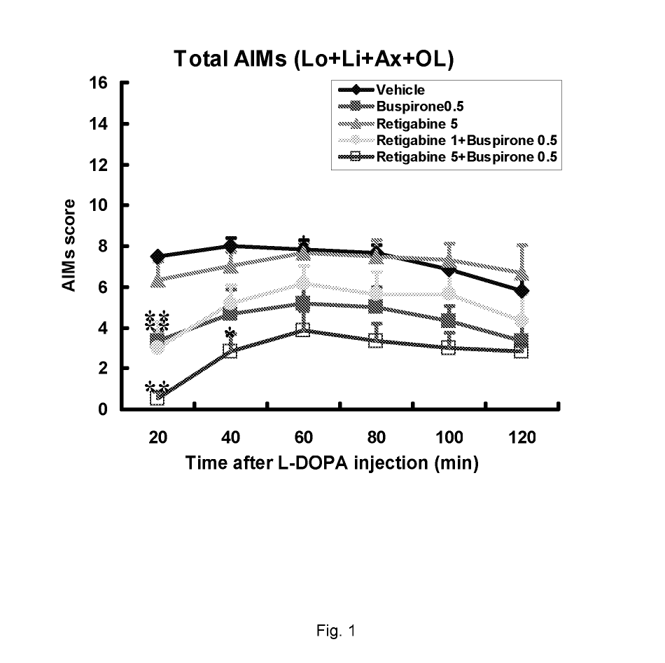

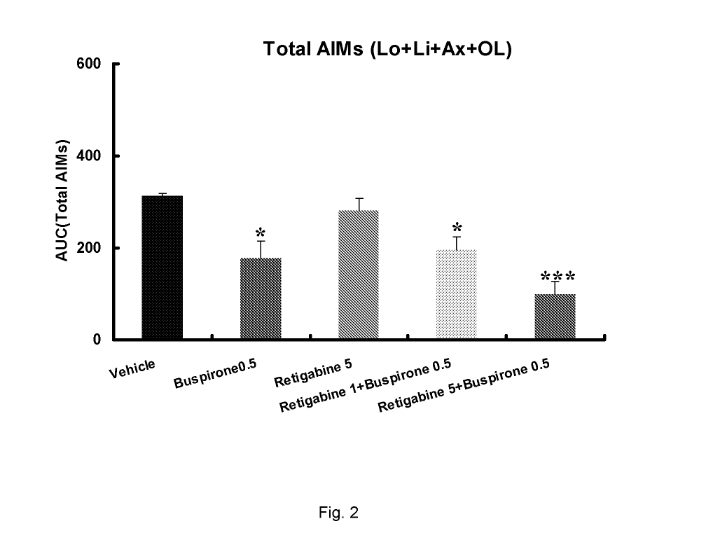

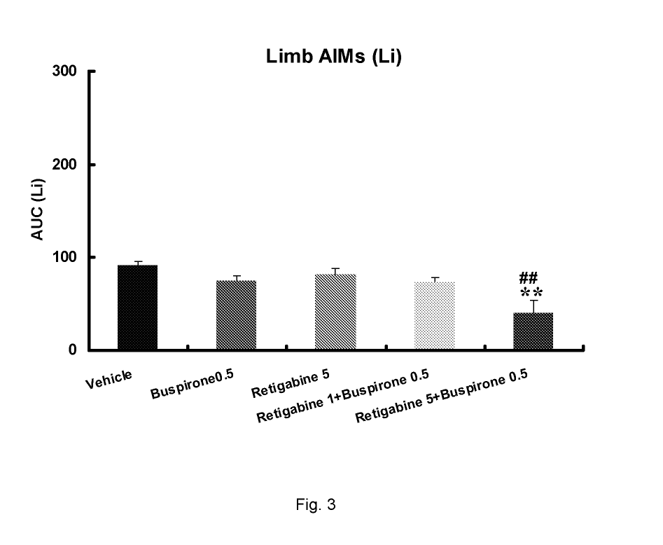

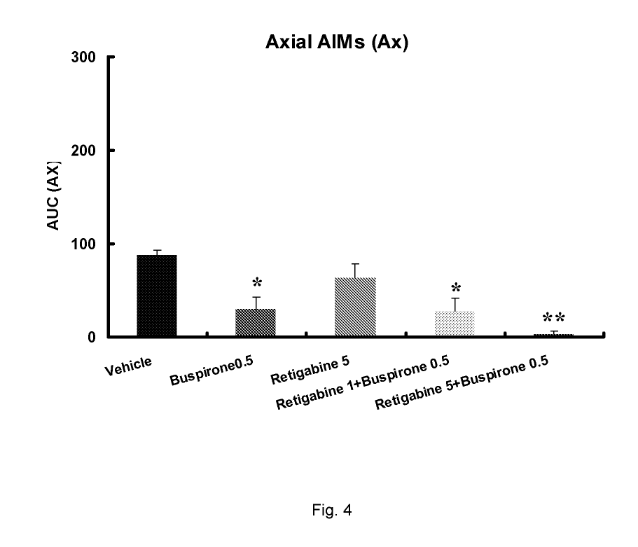

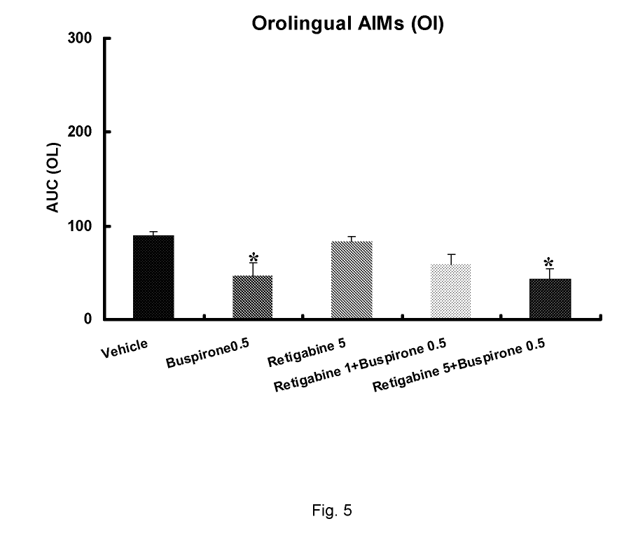

From the results shown in FIGS. 1, 2, 3, 4, and 5, it can be seen that the KCNQ channel activator compounds, the 5-HT1A receptor agonist compound and the combinations thereof used in the present study had different effects on AIM scores. It was found that retigabine alone (5 mg/kg i.p.) did not have an effect on AIM, while buspirone alone (0.5 mg/kg i.p.), and buspirone (0.5 mg/kg i.p.)) plus retigabine at lower doses (1 mg/kg i.p.) partially reduced AIM, while a combination of buspirone (0.5 mg/kg i.p.) and retigabine at higher doses (5 mg/kg ip) significantly reduced AIM. Furthermore it can be seen that the effects of the drugs and drug combinations on the different types of AIM's could be differentiated. Looking at the locomotive limb (Li) AIM scores it could be seen that combination of buspirone (0.5 mg/kg i.p.) and retigabine (5 mg/kg i.p.) significantly reduced AIM (Li) compared to vehicle, retigabine (5 mg/kg i.p.), buspirone (0.5 mg/kg i.p.), or buspirone (0.5 mg/kg i.p.) plus retigabine (1 mg/kg i.p.).

### Open Field Test

The open field test was used to determine the effects of the compounds buspirone and retigabine and combinations thereof on locomotor activity.

Species: 60 Sprague-Dawley male rats (180-220 g, bred in house, originally from SLAC Labortory Animal Co. Ltd) at 9-week of age.

Administration and dose regimen for the groups of animals (each comprising 10 rats):

Vehicle: 10% Tween 80, i.p., 30 min before test, n=10.
Buspirone 1: Buspirone (From Sigma, Cat. No. B7148, Lot. No. 042K1763Z) 1 mg/kg, i.p. 30 min before test, n=10.
Buspirone 2: Buspirone 2 mg/kg, i.p. 30 min before test, n=10.
Retigabine 10: Retigabine 10 mg/kg, i.p. 30 min before test, n=10.
Retigabine 10+Buspirone 1: Retigabine 10 mg/kg i.p. 5 min before buspirone 1 mg/kg, n=10.
Retigabine 10+Buspirone 2: Retigabine 10 mg/kg i.p. 5 min before buspirone 2 mg/kg, n=10.

Rats were put in open-field chambers (dimensions 40 cm×40 cm×40 cm) 30 minutes after dosing. After a 15 minutes habituation, locomotion were recorded and analyzed by Enthovision Video Tracking Software (Noldus Information Technology, Netherlands) for 60 minutes. All locomotor activities were done during dark phase and to eliminate olfactory cues, the arena was thoroughly cleaned with 70% v/v ethanol between each test.

Data statistics: The total locomotor activity is expressed as total moved distance (cm) and average velocity (cm/s) every 10 minutes during 60 minutes. The data were analyzed using One-Way ANOVA and the Tukey post-hoc test. The locomotor activity in six time point is expressed as moved distance (cm) and average velocity (cm/s) every 10 minutes. The data were analyzed using One-Way ANOVA and the Tukey post-hoc test in each time point.

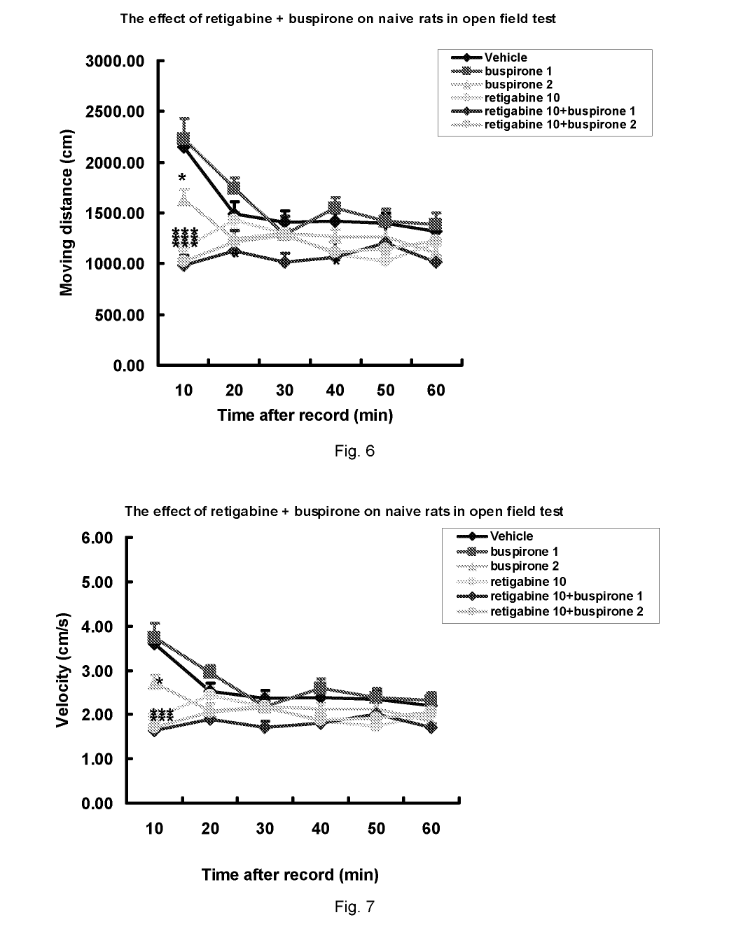

A time course of the moving distance (cm) and velocity (cm/s) during 60 minutes is shown in FIGS. 6 and 7. The data indicate that retigabine (10 mg/kg, i.p.) alone and retigabine (10 mg/kg, i.p.) combined with buspirone (1 mg/kg i.p. or 2 mg/kg, i.p.) initially (after 10 min) significantly inhibit the locomotor activity of rats in the open field test but that the effect disappears rapidly as there is no significant difference after 20 mins. Furthermore, the data indicate that combined administration of retigabine (10 mg/kg i.p.) with buspirone (1 mg/kg i.p. or 2 mg/kg i.p.) does not increase with respect to the locomotor activities or the sedative effects of retigabine administered alone (10 mg/kg i.p.).

### Example V

### Study of Prevention of L-DOPA Induced Movement Disorders

### Prevention:

In a prevention study rats are treated with L-DOPA methyl ester (6 mg/kg i.p. plus benserazide 15 mg/kg) in combination with buspirone (0.5-10 mg/kg/day) and flupirtine (0.5 mg/kg/day-20 mg/kg/day i.p.) given at the same time of L-DOPA, for 3 weeks. At the end of this treatment (treatment period 1), animals receive a low dose of apomorphine (0.02 mg/kg, s.c.) and tested for apomorphine-induced AIMs in order to investigate the sensitization state of the DA receptors. Treatments are then continued so that animals are treated only with L-DOPA for an additional two weeks (treatment period 2). Animals are injected daily and tested every second day for L-DOPA-induced dyskinesia throughout the experimental periods 1 and 2 and then sacrificed for HPLC analysis of DA, serotonin and metabolites.

### Example IV

### Studies of Motor Performance and Coordination in Rats Treated with Combinations of Compounds of the Present Invention

The rotarod test serves the purpose of detecting potential deleterious effects of the compounds studied on the rats' motor performance and coordination. In brief, the animals (30 SD male rats (180-220 g, bred in house, originally from SLAC Laboratory Animal Co. Ltd) at 9-week of age) are trained twice a day for a 3-day period. The rats are placed on the accelerating rod apparatus (Shanghai Jiliang, China) at an initial speed of 4 rotations per minute (rpm), with the speed increasing gradually and automatically to 40 rpm over 300 s. Each training trial is ended if the animal fell off or grips the device and spun around for two consecutive revolutions. The time that rat stayed on the rotarod is recorded. The staying duration recorded at last training trail is used as baseline. Rats are grouped according a randomly distribution of baseline. For the test session on the fourth day, the rats are evaluated on the rotarod with the same setting as above at 30 min after dosing. The rats are dosed with drugs as described below. Dosing and rotarod measurement are conducted by two scientists separately. Pentobarbital (15 mg/kg. i.p.) is used a as a positive control.

### Effects on Parkinson's Disease

### Stepping Test:

The stepping test (Schallert et al., 1992) is performed as described by Kirk et al., 2001 with little modifications. Briefly, the rat is held by the experimenter fixing its hind limbs with one hand and the forelimb not to be monitored with the other, while the unrestrained forepaw is touching the table. The number of adjusting steps is counted, while the rat is moved sideways along the table surface (90 cm in 5 s), in the forehand and backhand direction, for both forelimbs, and the average of the steps in the two directions is considered.

### Tacrine-Induced Tremulous Jaw Movements in Rats can be Used as an Experimental Model of Parkinsonian Tremor

Observations of tremulous jaw movements in rats are made in a 27×17.5×17 cm clear plexiglas chamber with a wire mesh floor. Tremulous jaw movements are defined as rapid vertical deflections of the lower jaw that resemble chewing but are not directed at any particular stimulus. Each individual deflection of the jaw is recorded using a mechanical hand counter. Jaw movements are recorded by an observer who is unaware of the experimental treatment conditions, and the observer is trained to demonstrate inter-rater reliability with a second observer over a number of pilot test sessions (r=0.92; P<0.05). To induce tremulous jaw movements, each rat receives an i.p. injection of 5.0 mg/kg of the anticholinesterase tacrine 10 min before testing. Rats are placed in the observation chamber immediately after tacrine injection for a 10-min habituation period.

### In Vivo Microdialysis and Behavior

Administration of L-DOPA to unilaterally 6-OHDA-lesioned rats induces abnormal involuntary movements (AIMs) and changes in concentrations of neurotransmitters in the brain. Using special methodologies it is possible to measure levels of such neurotransmitters (e.g. dopamine, gamma amino butyric acid (GABA), noradrenalin, serotonin) in different brain regions in freely moving rats that previously have been treated with 6-OHDA. This procedure allows for a direct comparison between central neurotransmitters and behavior and is a method used to determine mechanism of action and efficacy of compounds of the present invention.

Buspirone (1 mg/kg i.p. or 5 mg/kg i.p.) in combination with retigabine (5 mg/kg i.p) are shown to reduce central dopamine levels as determined by this method.

### PET Scanning

The levels of neurotransmitters and receptors for such neurotransmitters in different regions of the brain of animals and humans can be determined using PET scanning. Such procedures are useful to study levels of dopamine and dopamine receptors in healthy and disease animals and humans and thereby study effects of drug treatment of Parkinson's disease. Furthermore this procedure can be used to predict effects in humans from animal studies and are useful for predicting efficacy of drug combinations of the current invention. A commonly used PET tracer for studying dopamine levels in human volunteers, in patients suffering from Parkinson's disease and in animal models of Parkinson's disease is [¹¹C]raclopride. Raclopride is a ligand for the dopamine D2 and D3 receptors. Using PET scanning, this tracer allows for a determination of changes in extracellular dopamine levels caused by treatment with drugs and drug combinations.

The experimental setup testing various doses of retigabine (0.5-20 mg/kg i.p.) in combination with various doses of buspirone (0.5-20 mg/kg i. p.) shows that retigabine (5 mg/kg i.p.) in combination with buspirone (1 mg/kg i.p. or 5 mg/kg i.p.) reduces central dopamine levels as determined by this method.

### Example V

### A Study of the Combinations of a KCNQ Channel Activator and 5-HT1 Receptor Agonist of the Present Invention in a Model of Tardive Dyskinesia.

Groups of 6 male CD-1 mice weighing 36±10 g are used. On the first day of the study, one group receives the vehicle for reserpine (naïve control group), whereas the animals in the other groups are treated with two s.c. injections of resperpine (1 mg/kg, n=6) separated by 48 hours to induce tardive dyskinesia. Twenty-four hours after the last injection of reserpine (day 4), a KCNQ channel activator and a 5-HT1 receptor agonist are administered by i.p. injection. Behavioral assessment us carried out for 10 min, 1 hour after injection of the agents.

For the behavioral assessment, animals are individually placed in a plexiglass cage (13 cm×23 cm×13 cm). Mirrors are placed under the floor of the cage to permit observation of oral movements when the animals face away from the observer. After a 5-min period of habituation, the occurrence of vacuous chewing movements (VCM) is counted for a further 10-min period. VCM refers to a single mouth opening in the vertical plane not directed toward physical material. If VCM occurs during a period of grooming, they are nor taken into account. Values are presented as mean±SEM an unpaired Dunnett's test is applied for comparison between vehicle and compound-treated groups. Differences are considered significant at P<0.05.

The presence of significantly differences between rats administered with vehicle alone compared to a combination of a KCNQ channel activator and a 5-HT1 receptor agonist indicates that the combination has an effect on treatment of tardive dyskinesia.

### Example VI

### Treatment of Individuals Suffering from Movement Disorders

The following illustrates an example of the use of the compounds of the invention for treatment of patient suffering from LID:

A 69 years old woman has been diagnosed with PD 6 years ago and has since then been treated with L-DOPA/carbidopa (300/75 mg given in 3 divided doses). She has started to experience involuntary movements and is diagnosed with L-DOPA induced dyskinesia. The patient is treated with a combination of buspirone (20 mg/day) and a starting dose of retigabine (100 mg) administered orally three times a day. The dosage of buspirone is continued, while the dose of retigabine is increased by 150 mg/day every week, until the daily dose is 1100 mg/day (daily full dose). After 8 days of treatment on the daily full dose, the symptoms of dyskinesia are assessed by the scales Lang-Fahn Activities of Daily Living Dyskinesia scale, Clinical Global

Impression, Unified Parkinson's Disease Rating Scales as well as the Abnormal Involuntary Movement Scale (AIMS). The patient is continuously administered a combination of buspirone and retigabine.

### Example VII

### Evaluation of the 5-HT1 Receptor Agonist Buspirone and the KCNQ Positive Modulator Retigabine for Treatment of Movement Disorders Associated with Parkinson's Disease and LID; Effects of Sequential Dosing

The present study describes an evaluation of buspirone and retigabine in the 6-OHDA rat model using similar procedures as described in Example III.

85 SD male rats (220 g˜250 g, 8 weeks old) are unilaterally injected with 6-OHDA into medial forebrain bundle (MFB) to induce the Parkinson's disease (PD) model. 71 PD model rats (244 g˜319 g, 11 weeks old) are successfully created with the criteria of apomorphine induced rotations≧180/30 min.

After chronic L-DOPA treatment (8 mg/kg) plus benserazide (15 mg/kg, s.c.) on PD rats for 21 days, 45 LID model rats (352 g˜-468 g, 14 weeks old) are successfully created with the criteria of total AIM scores (Lo+Li+AX+OI)≧28 points. These rats are subsequently used for evaluation of effects of drugs and drug combinations using the following general procedure:

The dosing procedure is performed by appointed scientists who are not involved in the AIMs ratings. Test compounds are dosed at different time points before AIMs ratings. The L-DOPA (8 mg/kg)/Benserazide (15 mg/kg) mixture is dosed 10 min before AIMs ratings with s.c. injection (on each sides of the back of the rats).

AIMs ratings are performed in a quiet room by well-trained observers experimentally blind to the pharmacological treatment conditions. Rats are placed individually in transparent plastic cages without bedding material. Each rat is rated for 1 min every 20 min during the 190 min that follow the L-DOPA-injection. The subtypes of AIMs are classified into four subtypes: (1) locomotive AIMs (Lo), i.e., increased locomotion with contralateral side bias; (2) limb AIMs (Li), i.e., jerky and/or dystonic movements of the forelimb contralateral to the lesion; (3) axial AIMs (Ax), i.e., dystonic or choreiform torsion of the trunk and neck towards the side contralateral to the lesion; (4) orolingual AIMs (OI), i.e., twitching of orofacial muscles, and bursts of empty masticatory movements with protrusion of the tongue towards the side contralateral to the lesion. Each of the four subtypes is scored based on the duration and persistence of the dyskinetic behavior during the 1 min observation period. A rating scale of severity is from 0 to 4, where 0=absent, 1=present during less than half of the observation time, 2=present for more than half of the observation time, 3=present all the time but suppressible by external stimuli, and 4=present all the time and not suppressible by external stimuli.

### Example VII-a

### Effects of Differential Dosing of the Serotonin 5-HT1A Agonist Buspirone and the KCNQ Positive Modulator Retigabine on AIMs in the LID Model

Group setting and dosing: 5 ml/kg
1. Vehicle
2. Buspirone 0.2 mg/kg (11 min) sc.
3. Buspirone (0.2 mg/kg)/Retigabine (0.5 mg/kg) mixture (11 min) sc.
4. Buspirone (0.2 mg/kg)/Retigabine (5 mg/kg) mixture (11 min) sc.
5. Buspirone (0.2 mg/kg (11 min)+Retigabine 5 mg/kg (2 h) sc.
6. Buspirone (0.2 mg/kg (11 min)+Retigabine 5 mg/kg (5 h) sc.
7. Retigabine 5 mg/kg (11 min).

The drugs are administered at the above-cited doses by sub-cutaneous (s.c.) injections at different time-points before the AIM-test: Vehicle (1), buspirone alone (2), simultaneous administered buspirone/retigabine (3+4) and retigabine alone (7) are administered 11 minutes before AIM test. For sequential administration of buspirone and retigabine (5+6), retigabine is administered either 2 hours (5) or 5 hours (6) before the AIM test, while buspirone is administered 11 minutes before the AIM test (thus, retigabine is administered first).

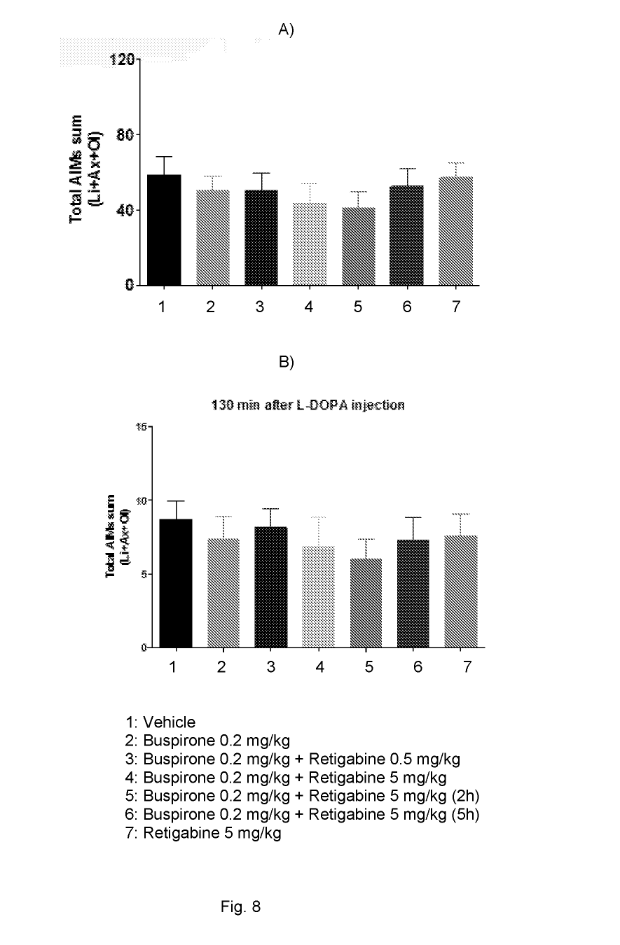

The combined results (all time points) are presented in FIG. 8A, and show a clear tendency for the combined effect of retigabine and buspirone when administered simultaneously (4) in that the total AIMs sum post-treatment is reduced. Furthermore, when retigabine is administered before buspirone (2 hrs before the AIM test vs. 11 minutes before the AIM test) it appears that at least the same and even improved beneficial effects are obtained for the sequential administration (5). At 5 hours difference (6) no effect is observed of the sequential administration. The same is the case for total AIMs at 130 minutes after L-DOPA injection (FIG. 8B).

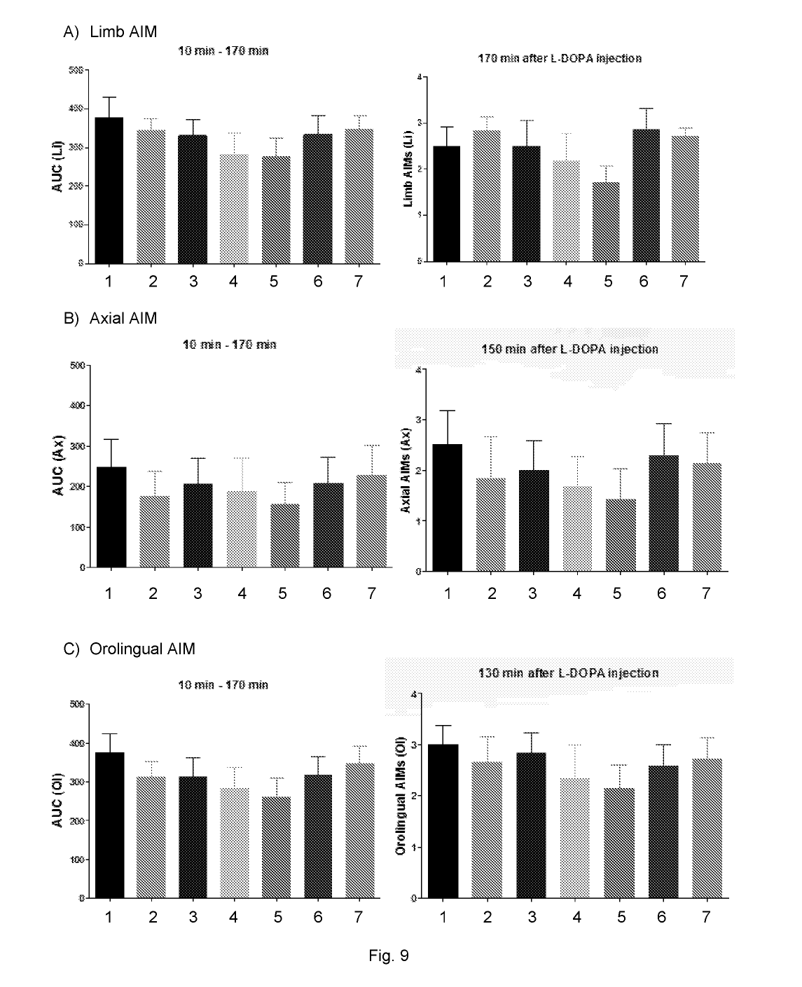

In FIG. 9, the effects of individual AIM a) Limb, B) Axial and C) Orolingual is presented at the combined time points 10-170 min, and at individual time points.

Thus, the data indicate that when retigabine is administered before buspirone, the sequential administration of each part of the combination will effectively reduce the total and individual AIM.

1.-46. (canceled)

47. A method for treatment, prevention or alleviation of movement disorders comprising:
one or more steps of administration of an effective amount of a pharmaceutical composition comprising a KCNQ channel activator, or a pharmaceutically acceptable derivative thereof, and
one or more steps of administration of an effective amount of a pharmaceutical composition comprising a serotonin 5-HT1 receptor agonist, or a pharmaceutical acceptable derivative thereof,
to an individual in need thereof.

48. The method according to claim 47, wherein the KCNQ activator is selected from the group consisting of i) an activator of one or more homomeric and/or heteromeric KCNQ channels each comprising one or more subunits selected from the group of Kv7.2, Kv7.3, Kv7.4 and Kv7.5; ii) an activator of one or more homomeric KCNQ channels; iii) an activator of one or more heteromeric KCNQ channels selected from the group of Kv7.2/3, Kv7.3/4 or Kv7.3/5 KCNQ channels; iv) an activator of one or more KCNQ channels expressed in the nervous system; v) an activator of one or more pre-synaptic, somatodendritic or post-synaptic KCNQ channels; and vi) an activator of one or more pre-synaptic or somatodendritic KCNQ channels.

49. The method according to claim 47, wherein the KCNQ activator is selected from the group consisting of retigabine, flupirtine, ICA-27243, BMS-204352 (racemic mixture maxipost), BMS-204352 (S enantiomer), Acrylamide (S)-1, Acrylamide (S)-2, diclofenac, meclofenamic acid, NH6, zinc pyrithione and ICA-105665.

50. The method according to claim 47, wherein the 5-HT1 receptor agonist is a 5-HT1A receptor agonist.

51. The method according to claim 50, wherein the 5-HT1A receptor agonist is selected from the group consisting of buspirone, tandospirone, gepirone, alnespirone, binospirone, ipsapirone, perospirone, befiradol, repinotanpiclozotan, osemozotan, flesinoxan, flibanserin and sarizotan, or a pharmaceutically acceptable derivative thereof.

52. The method according to claim 47, wherein the KCNQ activator is retigabine and the 5-HT1 receptor agonist is the 5-HT1A receptor agonist buspirone.

53. The method according to claim 47, wherein the 5-HT1 receptor agonist is an agonist of at least one or more of the receptors selected from the group consisting of: 5-HT1B, 5-HT1D and 5-HT1F receptors.

54. The method according to claim 47, wherein the KCNQ channel activator is administered in doses of 0.5 mg/day to 2000 mg/day; and wherein the 5-HT1 receptor agonist is administered in doses of 0.5 mg/day to 60 mg/day.

55. The method according to claim 47, wherein the KCNQ channel activator and the serotonin 5-HT1 receptor agonist, or pharmaceutically acceptable derivatives thereof, are comprised in the same pharmaceutical composition.

56. The method according to claim 47, wherein the KCNQ channel activator and the serotonin 5-HT1 receptor agonist are provided in separate formulations which are administered sequentially and/or separately.

57. The method according to claim 47, wherein the KCNQ channel activator is released or administered before, or before and during, the serotonin 5-HT1 receptor agonist.

58. The method according to claim 47, wherein the serotonin 5-HT1 receptor agonist is released or administered before, or before and during, the KCNQ channel activator.

59. The method according to claim 47, further comprising administration of one or more additional active ingredients, wherein said one or more additional active ingredients are released or administered by simultaneous, sequential or separate administration.

60. The method according to claim 59, wherein said one or more additional active ingredients are selected from the group consisting of: agents increasing the dopamine concentration in the synaptic cleft; dopamine; L-DOPA; dopamine receptor agonists; agents which ameliorate symptoms of Parkinson's disease or which are used for treatment of Parkinson's disease; decarboxylase inhibitors including carbidopa and benserazide; and catechol-O-methyl transferase (COMT) inhibitors including tolcaponeandentacapone.

61. The method according to claim 47, wherein the movement disorder is selected from the group consisting of a movement disorder associated with altered synaptic dopamine levels; Parkinson's disease; akathisia; tardive dyskinesia; dyskinesia associated with Parkinson's disease; and L-DOPA induced dyskinesia.

62. A pharmaceutical composition or kit of parts comprising a KCNQ channel activator and a serotonin 5-HT1A receptor agonist, or pharmaceutically acceptable derivatives thereof.

63. The pharmaceutical composition or kit of parts according to claim 62, wherein the KCNQ activator is selected from the group consisting of retigabine, flupirtine, ICA-27243, BMS-204352 (racemic mixture maxipost), BMS-204352 (S enantiomer), Acrylamide (S)-1, Acrylamide (S)-2, diclofenac, meclofenamic acid, NH6, zinc pyrithione and ICA-105665.

64. The pharmaceutical composition or kit of parts according to claim 62, wherein the 5-HT1 receptor agonist is a 5-HT1A receptor agonist, including but not limited to a 5-HT1A receptor agonist selected from the group consisting of buspirone, tandospirone, gepirone, alnespirone, binospirone, ipsapirone, perospirone, befiradol, repinotanpiclozotan, osemozotan, flesinoxan, flibanserin and sarizotan, or a pharmaceutically acceptable derivative thereof.

65. The pharmaceutical composition or kit of parts according to claim 62, wherein the KCNQ activator is retigabine and the 5-HT1 receptor agonist is the 5-HT1A receptor agonist buspirone.

66. The pharmaceutical composition or kit of parts according to claim 62, wherein the 5-HT1 receptor agonist is an agonist of at least one or more of the receptors selected from the group of 5-HT1B, 5-HT1D and 5-HT1F receptors.
